(19) United States
(12) Patent Application Publication
(10) Pub. No.: US 2026/0212730 A1
(43) Pub. Date: Jul. 23, 2026

(54) SPORTS BETTING AND GAMING PLATFORM WITH INTEGRATED AI-BASED FRAUD DETECTION

(71) Applicant: QOMPLX LLC, Reston, VA (US)

(72) Inventors: Jason Crabtree, Vienna, VA (US); Richard Kelley, Woodbridge, VA (US); Jason Hopper, Halifax (CA); David Park, Fairfax, VA (US)

(21) Appl. No.: 19/031,919

(22) Filed: Jan. 18, 2025

(51) Int. Cl. G07F 17/32; G06N 20/20; G06Q 50/34; G16H 10/60

(52) U.S. Cl. CPC G07F 17/3241; G06N 20/20; G06Q 50/34; G07F 17/3288; G16H 10/60

(57) ABSTRACT

A sports betting and fantasy gaming platform that incorporates AI-enabled fraud detection across multiple sports and competition levels. The platform integrates real-time game telemetry, social media data, betting patterns, and performance metrics to detect potential fraud through sophisticated pattern analysis. Sport-specific AI models, including generative adversarial networks and temporal convolutional networks, analyze player movements, team dynamics, and betting behaviors to identify anomalous patterns. The system enables event-oriented trading of fantasy assets and betting positions while maintaining regulatory compliance across jurisdictions. Advanced features include real-time integration with gaming platforms, collaborative team ownership structures, and automated hedging strategies. The platform’s modular design allows for customization based on sport type, regional gaming laws, and competition levels, from youth sports to professional leagues. Fraud detection capabilities extend beyond athletes to monitor team employees, officials, and other stakeholders, ensuring comprehensive protection of sports betting integrity.

### CROSS-REFERENCE TO RELATED APPLICATIONS

Priority is claimed in the application data sheet to the following patents or patent applications, each of which is expressly incorporated herein by reference in its entirety: None

### BACKGROUND OF THE INVENTION

### Field of the Art

The present invention is in the field of sports entertainment to include esports and live sports broadcast, interactive play, betting and gaming technology, and more particularly on AI- enabled feature enablement, fraud detection and risk management to enhance experience, support betting integrity and enable fair and compliant gaming operations across multiple sports and competition levels.

### Discussion of the State of the Art

Sports betting has rapidly expanded beyond traditional professional leagues into collegiate, amateur, and regional sports, creating new challenges for monitoring and preventing fraudulent activities. While professional athletes are subject to more rigorous scrutiny and often have significant financial and reputational incentives to avoid betting-related issues, participants at lower levels often lack similar protections, awareness, and maturity. This expansion of betting markets, combined with the rise of player-specific name, image and likeness deals, odds and performance-based wagers, and endorsements/advertising revenue opportunities has created unprecedented complexity in sports book and esports book operations to include fraud detection, mitigation, prevention, and investigation.

The sports betting market has experienced exponential growth with the NFL alone generating an estimated $35 billion in betting activity during the 2024-2025 season. This surge in betting volume, coupled with the recent expansion of legal sports betting to 34 states and Washington D.C., has created unprecedented challenges for maintaining sport reputation, betting integrity and preventing outright fraud and abuse. Industry analysts estimate that approximately 73.5 million Americans now have access to legal betting platforms, representing an increase of 27% from the previous year. This rapid expansion and massive transaction volume underscore the critical need for sophisticated player, team and league analytics, along with corresponding fraud detection and prevention systems that can operate at scale across multiple sports, jurisdictions and betting platforms and include data feeds from games, social media, practices, wearables, drones, other sensors, heath data, et cetera and manage the ever present tension between individuals' privacy and total access to reduce risks.

Recent incidents highlight the growing challenges in this space. For example, a baseball player's $99 bet resulted in $740,000 in lost potential earnings, demonstrating the severe consequences of inadequate monitoring systems. More concerning are cases like the scandal involving a player's interpreter admitting to stealing over $16 million from the player to cover his own gambling debts, illustrating how fraud extends beyond athletes to team employees and officials. Other events, like Yankees infamous 5th inning meltdown in the 2024 world series, might be legitimate but boggle the mind and the fanbase.

Current fraud detection systems face several limitations. First, they typically focus almost exclusively on professional sports, leaving lower-tier competitions including lucrative college sports with massive NIL programs vulnerable to manipulation and abuse. Second, traditional monitoring approaches often fail to detect subtle patterns of suspicious behavior across different stakeholders, including players (and their family members or close associates), recruiters, coaching staff, officials, and team employees. Third, existing systems struggle to adapt to regional variations in gaming regulations and betting cultures. Fourth, most fail to account for esports and fantasy leagues, related gameplay dynamics that bridge real-world and digital gaming and betting experiences.

The emergence of eSportsbooks and fantasy sports platforms have introduced substantial new vectors for potential fraud and manipulation of betting and other incentives with monetary or digital currency value. Many of these platforms offer odds on both player and team performance across multiple sports, creating complex interaction patterns that traditional monitoring systems cannot effectively track. The integration of real-world sports with fantasy leagues and eSports has further complicated fraud detection by introducing cross-platform, league and sport manipulation risks. Previous attempts to address these challenges have relied primarily on manual monitoring and basic statistical analysis or machine learning. However, these approaches have proven insufficient given the volume and complexity of modern sports betting data. Some systems have attempted to implement automated monitoring, but they typically operate in isolation, failing to integrate data sources such as social media activity, financial transactions, and real-time performance metrics.

Existing solutions generally lack the capability to analyze complex behavioral patterns across different sports and competition levels. They often fail to account for the unique characteristics of each sport and the varying levels of sophistication in betting activities from professional to amateur leagues. This limitation becomes particularly apparent when attempting to detect fraud in emerging betting markets or new forms of sports entertainment.

The rise of machine learning and artificial intelligence has created opportunities for more sophisticated fraud detection approaches. However, current implementations typically focus on narrow aspects of fraud detection rather than providing comprehensive, adaptable solutions that can evolve with changing patterns of fraudulent behavior. Moreover, existing systems rarely integrate real-time data collection with predictive modeling to enable proactive fraud prevention.

What is needed is an integrated sports betting and gaming platform that can effectively monitor and detect fraudulent activities across all levels of sports, while adapting to regional regulations and emerging forms of sports entertainment. Such a system should be capable of processing real-time data from multiple sources, analyzing complex behavioral patterns, and automatically responding to potential fraud indicators while maintaining compliance with local gaming regulations.

### SUMMARY OF THE INVENTION

Accordingly, the inventor has conceived and reduced to practice a comprehensive sports betting and fantasy gaming platform with integrated Al-enabled fraud detection capabilities that spans multiple sports, competition levels, and use cases, including hybrid real-fantasy gameplay and wagers (both direct and derivative). The platform combines ongoing unified batch and real- time analysis of sports performance with advanced fraud detection and fantasy gaming operations, creating a multi-stakeholder environment that serves athletes, fans, leagues, betting operators, regulators, gaming platforms, and other participants across the sports ecosystem. The platform implements various sport-specific statistical, machine learning, and Al models and simulations to analyze players, teams, skills, game dynamics, player and team performance, injuries, equipment and rule-based impacts to games, name, image and likeness-related value, and betting patterns, while enabling event-oriented trading of fantasy assets and betting positions. For example, in one embodiment for basketball, the system employs generative adversarial networks (GANs) through BasketballFlow-style modeling to analyze player movements and team dynamics, while in hockey, another embodiment employs temporal convolutional networks to achieve high accuracy in player tracking and identification. This sport-specific approach extends across multiple domains, from swimming's experimentation with biometric live data tracking analysis (which can be employed by the system across any sport) to football's tactical modeling and decision-making for players and coaches alike. With the addition of live telematics and biometric data, models and simulations can not only enable more robust detection of player effort versus outcome deviations, injuries, and game theory-like play dynamics, but also impact streaming rights, advertising, and other revenue opportunity values.

The system's capabilities extend beyond traditional sports betting to include innovative features such as real-time gaming platform integration, where actual sports or esports performance triggers events in video games, fantasy leagues, immersive AR/VR experiences, or combinations thereof. This creates dynamic ecosystems where personal athletic or other individual or team performances in specific scenarios can influence other content networks, including broadcast, streaming, and generative AI-enabled downstream processes subscribing to such feeds, alongside impacts to fantasy outcomes and other gaming experiences (e.g., player or character scores, skills, capabilities, injuries, or other parameter or entity tweaks). Note that personal or team biometric performance, even brainwave data (e.g., electroencephalogram data - EEG), might be made available via an integrated data marketplace or marketplace API system. The platform facilitates sophisticated risk management through event-oriented trading, allowing participants to hedge positions across multiple markets and manage exposure to factors like NIL rights and player health or conduct. The platform also incorporates comprehensive monitoring of non-player stakeholders, including team employees, contractors, and officials, while maintaining regulatory compliance (e.g., PII, health, security data handling) across jurisdictions (e.g., location of collection, location of processing, location of betting or contract venue). The system commonly leverages a mix of structured data, unstructured or raw data, semantically validated data (e.g., in a knowledge graph with a defined ontology), and event, timeseries, or graph data. This holistic approach enables the system to detect and prevent fraud at all levels of sports competition, from youth leagues to professional sports, while supporting the growing commercialization of sports through features like automated valuation adjustments, collaborative team ownership, and integrated fantasy gaming operations.

According to a preferred embodiment, a computing system for a sports and esports betting and gaming platform with integrated AI-enabled ecosystem wide fraud detection, the computing system comprising: one or more hardware processors configured for: training and refining a system of machine learning models on an ongoing basis, wherein each subsystem is a network of machine learning models, specialized for a particular sport or specific to a given team, position, or player; processing a plurality of sport, league, team, game and player data through a trained and entity fine-tuned machine learning model; generating a player, team, position, or league profile and a betting profile based on the sport, league, team, coach, or player data; cross-referencing the player profile, position profile, team profile, sport profile or the betting profile against a baseline profile, wherein deviations from the baseline profile are measured; generating a fraud alert when the measured deviations exceed a predetermined threshold; and transmitting the fraud alert to a regulator. According to another preferred embodiment, a similar profile and comparison may be created for a specific time window, play, sequence of plays, sequence of games, or other event sequence of real or hypothetical player and activity engagements.

According to another preferred embodiment, the system comprises one or more processors and memory storing instructions that, when executed, cause the system to instantiate a set of artificial intelligent agents, each trained to emulate specific players or entire teams using a combination of available historical datasets, biometric signals, EEG readings, and telemetric player-tracking information. These agents are not merely static representations but instead are continuously refined through unsupervised and reinforcement learning processes, enabling them to exhibit emergent behavior patterns that closely match real-world athletes. For instance, separate neural networks may be trained on past player performance data, gait analysis, heart rate variability, fatigue markers, and neural activity patterns captured via EEG, creating detailed, dynamic behavioral models capable of exhibiting player-specific tendencies, decision-making processes, and reaction times. In operation, the system can virtually simulate matchups, sequences of plays, or entire games that may never have occurred in reality, thus allowing for speculative analyses (e.g., pitting Michael Jordan against LeBron James with both in their prime). The system can also repeatedly replay pivotal historical scenarios with slightly altered conditions-such as changing referee decisions, environmental factors, or opponent strategies to produce a distribution of outcomes and probabilities. Through this analysis, it can evaluate the likelihood that a certain outcome was influenced by irregular factors rather than genuine competitive variance. Such outcomes can be aggregated to form robust probabilistic models, identifying patterns that could be suggestive of game-fixing or other fraudulent activities. To accomplish this, the system integrates multi-tiered neural modules, each trained at different levels of granularity-some focusing on mechanical skill execution (e.g., a player's signature jump-shot motion), while others emphasize higher-level strategic decisions or emotional states inferred from biometric data. By integrating these modules into a single coherent agent, the system produces a versatile simulation engine that can adapt to novel conditions or constraints and generate realistic, data-driven outcomes. A global orchestrator module can coordinate multiple Al agents in a simulated environment, running high-fidelity trials efficiently at scale. The results of these simulations-such as distributions of outcomes or probability scores for certain suspicious events-can then be fed into a fraud detection subsystem, further refining the detection models and alerting regulators when statistical anomalies consistent with potential manipulation surpass predetermined thresholds. This embodiment extends the analytical and predictive capabilities of the underlying platform, enhancing not only the accuracy of betting and game outcome predictions but also significantly strengthening the detection and prevention of unethical behaviors. By leveraging a broad range of data modalities and advanced Al simulation methods, the system broadens its scope to encompass practically any desired scenario, from untried matchups between legendary players to investigative replays of historically significant, potentially compromised games. This approach provides regulators, teams, and leagues with a powerful, data-rich decision support tool capable of generating highly detailed insights previously beyond reach

According to a preferred embodiment, a computer-implemented method for a sports betting and gaming platform with integrated AI-enabled fraud detection, the computer- implemented method comprising: training a network of machine learning models, wherein each machine learning model is specialized for a particular sport; processing a plurality of sport and player data through a trained machine learning model; generating a player profile and a betting profile based on the sport and player data; cross-referencing the player profile or the betting profile against a baseline profile, wherein deviations from the baseline profile are measured; generating a fraud alert when the measured deviations exceed a predetermined threshold; and transmitting the fraud alert to a regulator, is disclosed

According to a preferred embodiment, a system for a sports betting and gaming platform with integrated AI-enabled fraud detection, comprising one or more computers with executable instructions that, when executed, cause the system to: train a network of machine learning models, wherein each machine learning model is specialized for a particular sport; process a plurality of sport and player data through a trained machine learning model; generate a player profile, better profile, and a betting profile of all bets based on the sport and player data; cross- reference the player profile, better profile, or the betting profile against a baseline profile, wherein deviations from the baseline profile are measured; generate a fraud alert when the measured deviations exceed a predetermined threshold; and transmit the fraud alert to a regulator, is disclosed.

In an aspect of an embodiment, the sport and player data comprises game telemetry, betting history, social media content, biometric information, kinesiology information, personal health database information (e.g., sleep, surgeries, blood panels, hormone levels), and facility- specific environmental factors.

According to another preferred embodiment, the system's data processing capabilities extend to detecting subtle biomechanical deviations or kinesiology-based variances in player movements. For example, a basketball player's shooting motion may be deconstructed into a multi-dimensional vector encoding joint angles, release timing, wrist tension, vertical leap height, rotational torque of the torso, and follow-through consistency. By comparing these parameters against a historical baseline, the system can detect suspicious irregularities if the player's shot trajectory angle abruptly deviates by a few degrees in a critical playoff game, or if their typical knee flexion at the point of release is reduced by a statistically significant margin. In baseball, the system may track a pitcher's elbow extension and grip pressure, flagging when the pitcher's signature curveball spin rate suddenly diminishes without a biomechanical or injury- related explanation, potentially indicating deliberate underperformance.

Another embodiment focuses on neurophysiological signals, integrating EEG or heart rate variability data where available. For instance, if a player known for maintaining a consistent calm state in high-pressure situations exhibits abnormal neural arousal patterns or erratic heart rate fluctuations during a crucial moment-directly contrasting established baselines-this may indicate external influence or intentional manipulation. The system's machine learning models, trained on historical neurophysiological responses, can assign a confidence score indicating whether a player's changed biometric signature is outside expected performance variance, thereby triggering a fraud alert. Further embodiments incorporate environmental and equipment telemetry data. Consider a soccer striker who normally accelerates toward the ball with a particular foot strike cadence and maintains an optimal center of gravity before shooting. If subtle deviations arise, such as a repeated millisecond delay in triggering forward acceleration or a newly detected uneven pressure distribution in their boot's smart insoles, the system can highlight these deviations. Coupled with contextual factors-like unusual betting patterns on that player's performance-these mechanical anomalies may serve as early indicators of suspicious behavior. Similarly, for golf or tennis, slight deviations in wrist orientation, racket speed at impact, or unexpected grip-pressure patterns against a stable baseline could suggest deliberate underperformance to manipulate outcome-based bets. Moreover, the system can scale across multiple players and plays, enabling scenario-level detection. For example, in American football, the system may analyze an entire drive sequence, identifying when several linemen simultaneously display joint-angle reductions in their stances, reduced explosive force off the line, or atypical play recognition patterns. Such synchronous, team-wide mechanical anomalies-corroborated by suspicious movement velocities detected through player-tracking chips-could indicate that these deviations are not random but potentially orchestrated. These findings, when combined with unusual real-time betting activity, lead the system to generate a high-confidence fraud alert, providing regulators with data-rich evidence of questionable play integrity and associated reasoning (e.g., simulation runs and model outputs).

In an aspect of an embodiment, the profiles comprise performance metrics, team dynamics, historical patterns, and network interaction analysis across multiple competition levels. This may also include biometric data, personal finance information, social media network information to identify friends and family. Historical betting data of the players, coaches, staff, or betters may also be used to establish a personal or group specific or team specific or other categorical risk profile based on characteristic clustering around networks (e.g., snapshotted over time), network-based signals and models (e.g., using Graph Neural Networks), or similarities (e.g., groups of players that might resemble skill/personality/team mixes of prior groups).

In an aspect of an embodiment, the system may generate simulated video content, multimedia content, or video game content for consumption or interactive events. This capability enables bettors to see custom replays, verify potential fouls, and analyze the probabilities of officiating decisions (e.g., comparing an on-field touchdown ruling versus a replay overturn). The system's content generation capabilities build upon established frameworks in generative Al for video synthesis while incorporating sport-specific knowledge and rules. Drawing from recent advances in multimodal learning demonstrated in works like Mirasol-3B, the system employs separate autoregressive models to handle time-synchronized modalities (video and audio) and contextual information. This approach efficiently processes high-volume video and audio inputs by partitioning them into consecutive snippets and using a specialized Combiner mechanism to fuse audio-visual features into compact, expressive representations. This is particularly valuable for processing lengthy game sequences and maintaining temporal coherence in sports replays. The system also leverages advances in text-to-video generation, as demonstrated by models like Sora, which enable the simulation of realistic physical worlds from textual descriptions. This capability allows the system to generate high-fidelity visualizations of both historical and hypothetical game scenarios, potentially transforming applications across sports analysis, training, and entertainment. The integration of such technology enables detailed analysis of player movements, officiating decisions, and game-changing moments while maintaining broadcast-quality visual fidelity.

In aspect of an embodiment, the network of machine learning models includes generative adversarial networks, temporal convolutional networks, graph neural networks, variational autoencoders, and graph attention networks.

In an aspect of an embodiment, the one or more hardware processors are further configured for: accessing a personal health database (PHDB) containing historical health records, injury data, and recovery patterns or reinjury rates; preprocessing the PHDB information to standardize health metrics across different sports and competition levels; processing the preprocessed PHDB information through the trained machine learning models that and incorporating the data into the player profile, group profile, team profile, league, or betting profile.

### BRIEF DESCRIPTION OF THE DRAWING FIGURES

FIG. 1 is a block diagram illustrating an exemplary system architecture for a sports betting and gaming platform with integrated AI-based fraud detection.

FIG. 2 is a block diagram illustrating a component for a sports betting and gaming platform with integrated AI-based fraud detection, a fraud detection subsystem.

FIG. 3 is a block diagram illustrating a component for a sports betting and gaming platform with integrated AI-based fraud detection, a machine learning training subsystem.

FIG. 4 is a block diagram illustrating a component for a sports betting and gaming platform with integrated AI-based fraud detection, a machine learning network.

FIG. 5 is a block diagram illustrating a component for a sports betting and gaming platform with integrated AI-based fraud detection, a hockey generative Al.

FIG. 6 is a block diagram illustrating a component for a sports betting and gaming platform with integrated AI-based fraud detection, a basketball generative Al.

FIG. 7 is a block diagram illustrating a component for a sports betting and gaming platform with integrated AI-based fraud detection, a swimming generative Al.

FIG. 8 is a high-level architecture diagram of an exemplary system for using personal health databases (PHDBs), according to an aspect of the invention.

FIG. 9 is a system diagram showing an exemplary arrangement of cloud-based PHDB- related computing, according to an aspect of the invention.

FIG. 10 is a system diagram showing an exemplary arrangement of a PHDB computing server or service with registration controls and third-party service integration, according to an aspect of the invention.

FIG. 11 is a system diagram showing an exemplary arrangement of a PHDB orchestration service according to an aspect of the invention.

FIG. 12 is a block diagram illustrating a component for a sports betting and gaming platform with integrated AI-based fraud detection, a sports betting and fantasy gaming platform.

FIG. 13 is a flow diagram illustrating an exemplary method for a sports betting and gaming platform with integrated AI-based fraud detection.

FIG. 14 is a flow diagram illustrating an exemplary method for detecting fraudulent bets made by players or game staff.

FIG. 15 is a flow diagram illustrating an exemplary method for generating player profiles and betting profiles.

FIG. 16 is a flow diagram illustrating an exemplary method for integrating real time sports data into video games or sports betting platforms.

FIG. 17 is a flow diagram illustrating an exemplary method for operating and managing a fantasy team platform.

FIG. 18 illustrates an exemplary computing environment on which an embodiment described herein may be implemented.

FIG. 19 is a flow diagram illustrating an exemplary method for generating and analyzing hypothetical game scenarios using AI-enabled simulation.

### DETAILED DESCRIPTION OF THE INVENTION

A comprehensive sports betting and fantasy gaming platform with integrated AI-driven fraud detection capabilities that spans multiple sports and competition levels. The platform leverages distributed computing architecture to process real-time game telemetry, social media data, betting patterns, and performance metrics through sport-specific Al models for fraud detection and automated response. The system incorporates generative adversarial networks, temporal convolutional networks, and graph attention networks to analyze player movements, team dynamics, and betting behaviors while enabling event-oriented trading of fantasy assets and betting positions.

The platform implements a modular architecture that integrates real-time data processing, machine learning-based analysis, and automated response systems across multiple sports contexts. Sport-specific generative Al modules analyze game dynamics and performance patterns while maintaining regulatory compliance across jurisdictions. The system enables enterprises to construct sophisticated betting and gaming operations with integrated fraud detection, leveraging advanced features such as collaborative team ownership, automated or semi-automated hedging strategies, and real-time gaming and experience platform integration. The platform facilitates comprehensive monitoring of athletes, team employees, officials, and other stakeholders (e.g., owners) through dynamic risk assessment and pattern recognition, supported by automated alert systems and regulatory compliance frameworks. This platform also supports fraud detection in fantasy leagues and other organizations that may abstract a particular sport, team, or player. The platform also supports prediction markets and alternative derivatives and contracts involving financial or other instruments of value.

One or more different aspects may be described in the present application. Further, for one or more of the aspects described herein, numerous alternative arrangements may be described; it should be appreciated that these are presented for illustrative purposes only and are not limiting of the aspects contained herein or the claims presented herein in any way. One or more of the arrangements may be widely applicable to numerous aspects, as may be readily apparent from the disclosure. In general, arrangements are described in sufficient detail to enable those skilled in the art to practice one or more of the aspects, and it should be appreciated that other arrangements may be utilized and that structural, logical, software, electrical and other changes may be made without departing from the scope of the particular aspects. Particular features of one or more of the aspects described herein may be described with reference to one or more particular aspects or figures that form a part of the present disclosure, and in which are shown, by way of illustration, specific arrangements of one or more of the aspects. It should be appreciated, however, that such features are not limited to usage in the one or more particular aspects or figures with reference to which they are described. The present disclosure is neither a literal description of all arrangements of one or more of the aspects nor a listing of features of one or more of the aspects that must be present in all arrangements.

Headings of sections provided in this patent application and the title of this patent application are for convenience only, and are not to be taken as limiting the disclosure in any way.

Devices that are in communication with each other need not be in continuous communication with each other, unless expressly specified otherwise. In addition, devices that are in communication with each other may communicate directly or indirectly through one or more communication means or intermediaries, logical or physical.

A description of an aspect with several components in communication with each other does not imply that all such components are required. To the contrary, a variety of optional components may be described to illustrate a wide variety of possible aspects and in order to more fully illustrate one or more aspects. Similarly, although process steps, method steps, algorithms or the like may be described in a sequential order, such processes, methods and algorithms may generally be configured to work in alternate orders, unless specifically stated to the contrary. In other words, any sequence or order of steps that may be described in this patent application does not, in and of itself, indicate a requirement that the steps be performed in that order. The steps of described processes may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to one or more of the aspects, and does not imply that the illustrated process is preferred. Also, steps are generally described once per aspect, but this does not mean they must occur once, or that they may only occur once each time a process, method, or algorithm is carried out or executed. Some steps may be omitted in some aspects or some occurrences, or some steps may be executed more than once in a given aspect or occurrence.

When a single device or article is described herein, it will be readily apparent that more than one device or article may be used in place of a single device or article. Similarly, where more than one device or article is described herein, it will be readily apparent that a single device or article may be used in place of the more than one device or article.

The functionality or the features of a device may be alternatively embodied by one or more other devices that are not explicitly described as having such functionality or features. Thus, other aspects need not include the device itself.

Techniques and mechanisms described or referenced herein will sometimes be described in singular form for clarity. However, it should be appreciated that particular aspects may include multiple iterations of a technique or multiple instantiations of a mechanism unless noted otherwise. Process descriptions or blocks in figures should be understood as representing modules, segments, or portions of code which include one or more executable instructions for implementing specific logical functions or steps in the process. Alternate implementations are included within the scope of various aspects in which, for example, functions may be executed out of order from that shown or discussed, including substantially concurrently or in reverse order, depending on the functionality involved, as would be understood by those having ordinary skill in the art.

### Conceptual Architecture

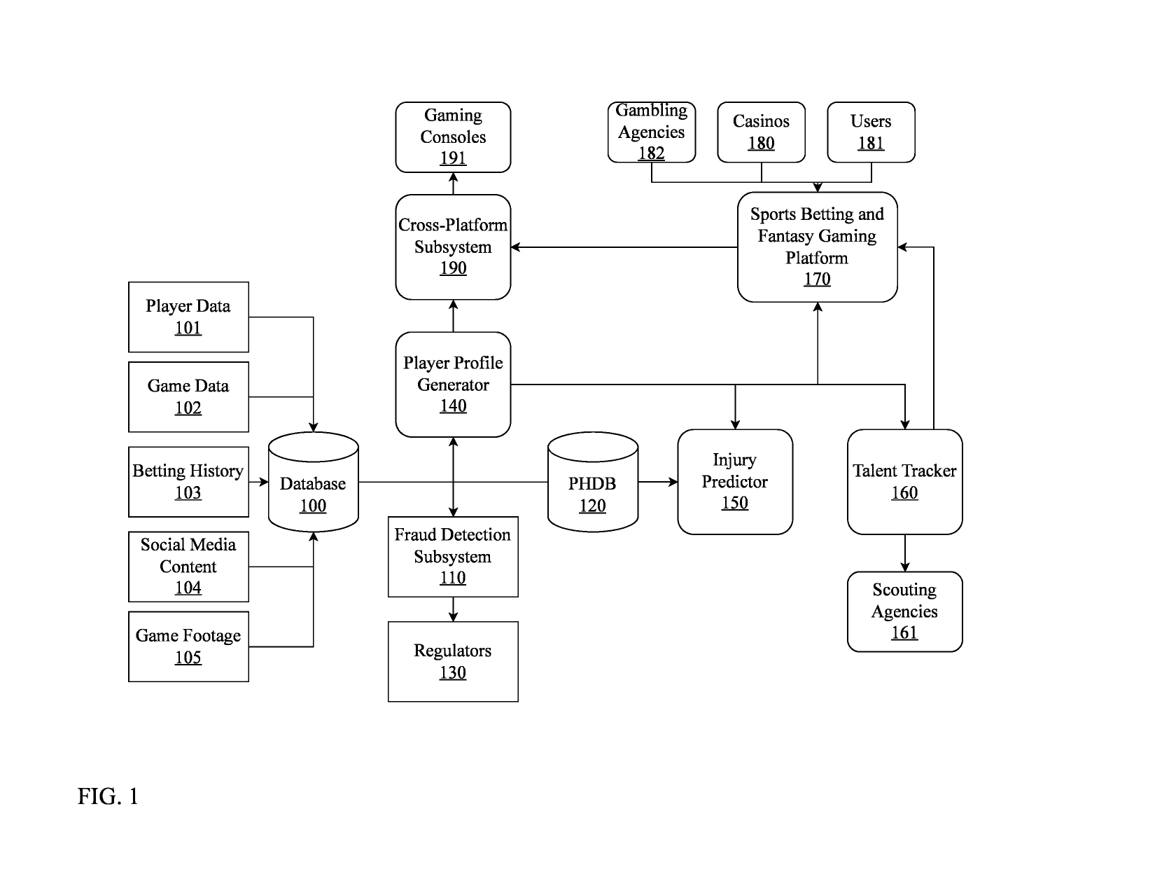

FIG. 1 is a block diagram illustrating an exemplary system architecture for a sports betting and gaming platform with integrated AI-based fraud detection. The platform centers on a database 100 that aggregates and processes multiple data streams to enable comprehensive fraud detection and sports gaming functionality. The database 100 is capable of receiving and storing player data 101, which includes but is not limited to biometric information, performance statistics, and historical records across various sports and competition levels. This player data is contextualized against game data 102 which may contain real-time telemetry, environmental factors, and venue-specific information that may influence athlete performance. For example, in swimming events, the game data may include pool depth measurements and water dynamics that affect performance outcomes.

Database 100 may also incorporate betting history 103 and social media content 104 to build comprehensive risk profiles of players, officials, and other stakeholders. The betting history provides patterns of wagering activity that, when analyzed alongside social media content, can reveal potential fraud indicators. For instance, as demonstrated in a FanDuel scandal involving team employees, unusual betting patterns combined with financial transactions can signal fraudulent activity. Game footage 105 feeds into the database, enabling real-time analysis of player movements and game events through computer vision and motion tracking technologies by using machine learning models such as a convolutional neural network (CNN).

A fraud detection subsystem 110 processes this aggregated data using a plurality of advanced Al models. For example, in hockey applications, the system may employ machine learning models such as convolutional neural networks (CNNs) and spatial temporal analysis, making it capable of tracking player posture, position, velocity, identifying players via facial recognition or jersey logo/number identification, puck, ball, or other sport equipment to track position, speed, and direction. These measurements extend from players to spectators of live matches to identify and track both individual spectators as well as the crowd as a whole. Crowd reactions can be quantized and compared to expected benchmark parameters, as can individual spectators be tracked across multiple events to estimate reactions such as surprise. These parameters can be tracked using a combination of many machine learning models that are each trained for purpose and integrated together to achieve a complete model of events. This subsystem may integrate with a personal health database (PHDB) 120 to incorporate health- related factors into fraud detection algorithms, while maintaining compliance with regulators 130 across different jurisdictions.

A player profile generator 140 creates detailed profiles incorporating performance data, financial data, health data, betting patterns, and social media activity. These profiles feed into both an injury predictor 150 and talent tracker 160, which help assess the likelihood of performance anomalies that could indicate fraudulent activity. The talent tracker shares data with scouting agencies 161, enabling verification of legitimate performance improvements versus suspicious patterns.

The sports betting and fantasy gaming platform 170 serves as the central interface for users 181, casinos 180, and gambling agencies 182. This platform implements real-time odds generation and risk assessment based on the analyzed data streams. The system's architecture includes a cross-platform subsystem 190 that enables integration with gaming consoles 191, allowing for the implementation of features like real-time performance updates in video games based on actual sports events.

Through this integrated architecture, the platform can detect and respond to potential fraud across multiple stakeholder groups, from professional athletes to team employees and officials. The system's ability to process diverse data streams and adapt to different sports and competition levels makes it particularly effective at identifying suspicious patterns that might be missed by traditional monitoring systems. For example, the platform can analyze a basketball player's movement patterns through machine learning systems such as CNNs, GANs, diffusion probabilistic models, or other generative Al models, and compare them against historical performance data to detect intentional underperformance, while simultaneously monitoring associated betting patterns for anomalies.

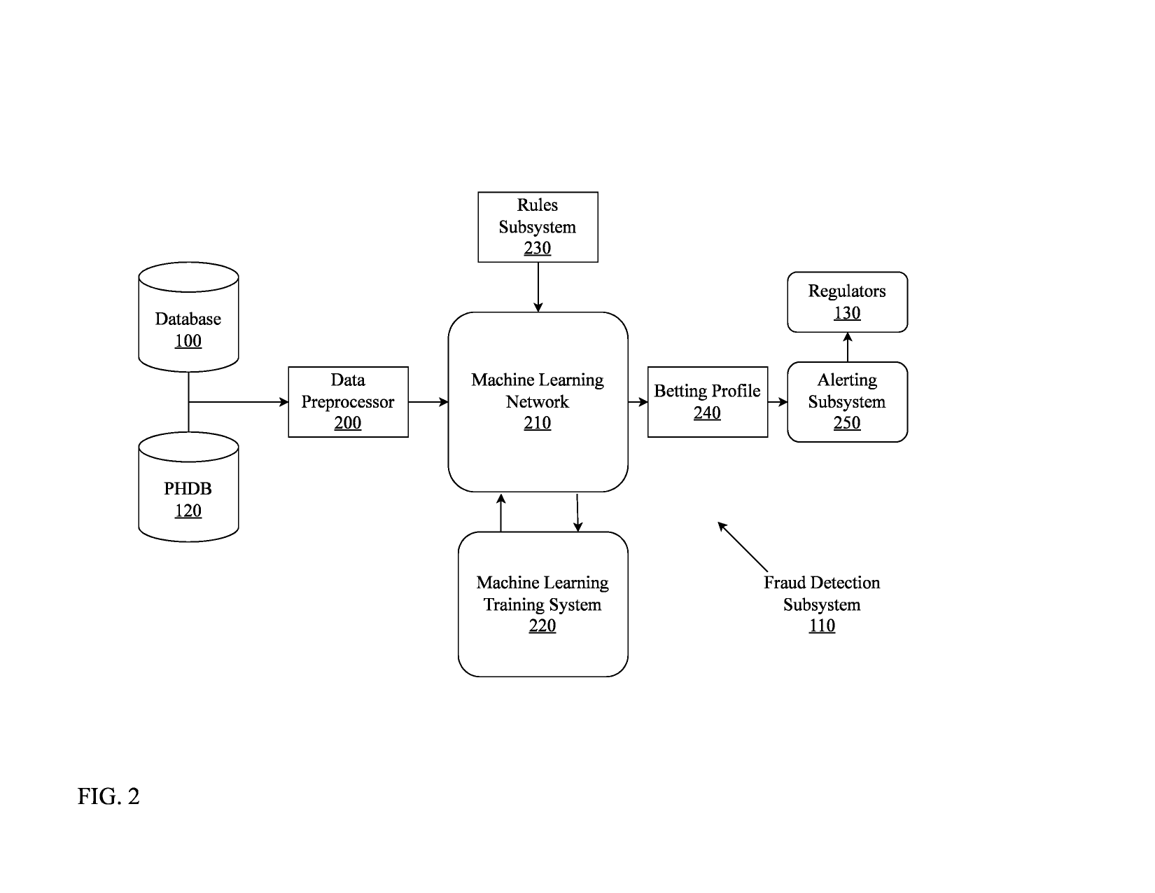

FIG. 2 is a block diagram illustrating a component for a sports betting and gaming platform with integrated AI-based fraud detection, a fraud detection subsystem. At the entry point, a data preprocessor 200 standardizes and normalizes incoming data streams for analysis. This preprocessing handles diverse inputs ranging from biometric data such as heart rate and fatigue indicators in swimming competitions to complex game telemetry such as ball or puck movement tracking and betting patterns.

The preprocessed data feeds into a machine learning network 210 that employs various Al models tailored to specific sports. For example, in basketball analysis, the system utilizes generative adversarial networks CNNs, GANs, diffusion probabilistic models, or other generative Al models through implementations like BasketballFlow to simulate and analyze gameplay scenarios, enabling the detection of anomalous behaviors that may indicate fraudulent activity. In hockey applications, the network incorporates temporal convolutional networks for player tracking and identification, achieving high accuracy in real-time monitoring of player movements and team dynamics.

A machine learning training system 220 continuously refines these models using new data and detected fraud patterns. The training system may incorporate techniques like Upper Confidence bounds applied to Trees (UTC) or Monte Carlo Tree Search (MCTS) algorithms for state transition modeling, allowing for dynamic lookback analysis, current state estimation, and look ahead forecasting with variable branching factors based on ongoing gameplay. This adaptive approach ensures the system can identify emerging fraud patterns while reducing false positives.

A rules subsystem 230 maintains and enforces compliance with regulatory requirements from regulators 130 across different jurisdictions. This component is particularly useful when monitoring betting activities across various competition levels, from professional leagues to amateur sports, where regulatory requirements may differ significantly. The rules subsystem also adapts to sport-specific regulations and local gaming commission rules, ensuring consistent fraud detection while maintaining compliance with regional requirements.

A betting profile component 240 generates comprehensive risk assessments by analyzing patterns in betting activity, social media presence, and historical performance data. This profiling is particularly effective in detecting potential fraud from non-player sources, as demonstrated in cases like the FanDuel scandal where a team employee exploited insider access. The betting profile component can identify suspicious patterns such as unusual betting volumes or correlated betting activities across multiple accounts.

Fraud detection subsystem 110 employs pattern recognition through its machine learning network 210 to identify suspicious betting activities across multiple accounts and markets. This may include machine learning models such as graph attention networks, generative Al models, or neural networks combined with portfolio optimization strategies. As betting data flows through the data preprocessor 200, the system analyzes bet timing, volumes, and patterns, comparing them against established behavioral models in the database 100. For instance, if multiple accounts demonstrate coordinated betting activities or unusual volume spikes on specific player performance metrics, the betting profile generator 430 flags these correlated patterns for detailed analysis through the plurality of Al architectures.

The data post processor 450 aggregates these findings while incorporating additional context from betting history 103, social media content 104, and game footage 105 to build comprehensive risk assessments. In one embodiment, a game footage predictor 440 examines whether betting patterns align with actual game events and performance metrics, helping distinguish between legitimate betting trends and potential fraud schemes. When suspicious patterns are detected, such as multiple accounts placing similar high-stakes bets within a short timeframe or coordinated betting across different locations, the alerting subsystem 250 triggers notifications while maintaining compliance with regulatory requirements through the rules subsystem 230. This approach enables the fraud detection subsystem to identify complex fraud schemes that might otherwise go unnoticed when analyzing accounts in isolation.

The fraud detection subsystem 110 continuously cross-references generated profiles against established baseline profiles stored in database 100. The system measures deviations using dynamic state transition modeling with variable branching factors, allowing for sophisticated pattern analysis across multiple dimensions. For instance, the system can simultaneously evaluate a basketball player's movement patterns through CNNs, GANs, diffusion probabilistic models, or other generative Al models while monitoring associated betting activities and social media interactions to build a comprehensive risk assessment.

The system establishes baseline profiles through a multi-stage data aggregation and analysis process. Data preprocessor 200 ingests historical data from multiple sources: game performance data from platforms like GameChanger and TeamSnap for youth and amateur levels, professional statistics from database 100, betting history 103, social media content 104, and game footage 105. For individual athletes, the player profile generator 140 incorporates additional data streams including PHDB 120 health records, competition results across multiple levels, and facility-specific performance data (such as pool depths in swimming or venue characteristics that might affect performance). Machine learning training system 220 processes this historical data through sport-specific Al modules to establish normal operating parameters for different competition levels, sports, and venues.

Profile generation occurs in real-time as the system processes new data through its sport- specific architectures. For basketball, the generative Al 410 may employ CNNs, GANs, diffusion probabilistic models, or other generative Al models to analyze player movements and team dynamics, while in hockey, the generative Al 400 may use temporal CNNs to achieve accuracy in player tracking. The betting profile generator 430 creates comprehensive betting profiles by analyzing patterns across betting history 103, current market activities monitored by market monitor 1280, and associated performance data. These betting profiles incorporate analysis of user behavior through user manager 1240, including patterns of bet timing, volume, and distribution across different markets and accounts. The analytics subsystem 1270 continuously processes new telemetry data against these established profiles, while the event processor 1230 validates real-time performance data against historical patterns.

The system maintains dynamic baselines that evolve based on new data and validated patterns. The machine learning network 210 employs transfer learning techniques to adapt baselines across different competition levels while maintaining sport-specific characteristics. The talent tracker 160 aids in establishing appropriate performance expectations, helping distinguish legitimate exceptional performance (like that of elite athletes), or underperformance, from suspicious patterns. The compliance monitor 1260 ensures these evolving baselines maintain alignment with regulatory requirements from regulators 130, while the rules subsystem 230 enforces sport-specific and jurisdiction-specific compliance standards. This comprehensive approach enables the system to maintain accurate, current baselines while accounting for legitimate performance variations and regulatory requirements across different sports and competition levels.

When measured deviations exceed configured or calculated thresholds, the alerting subsystem 250 automatically generates fraud alerts. These thresholds are dynamically adjusted based on multiple factors including competition level, venue characteristics, and regulatory requirements maintained by rules subsystem 230. The system transmits generated alerts to regulators 130 through integration subsystem 1250, which maintains secure communication channels with regulatory authorities across different jurisdictions. This automated alert system enables rapid response to potential fraud indicators while ensuring compliance with varying regulatory requirements across different sports and competition levels.

An alerting subsystem 250 generates real-time notifications when suspicious patterns are detected. These alerts provide the full context of why the alert was generated, and can trigger various automated responses, from temporarily suspending betting activity to initiating more detailed investigations. For instance, if a swimmer's performance deviates significantly from their historical patterns while unusual betting activity is detected, the system can automatically flag the event for review and notify relevant stakeholders.

This architecture enables pattern recognition across multiple data dimensions. For example, when analyzing a basketball player's performance, the system can simultaneously evaluate their movement patterns through GANs, monitor associated betting activities, and cross- reference social media and financial transactions to build a comprehensive risk assessment. The system's ability to process and correlate these diverse data streams in real-time, while maintaining regulatory compliance, makes it particularly effective at detecting complex fraud schemes that might evade traditional monitoring systems.

The feedback loop between the alerting subsystem 250, machine learning training system 220, and machine learning network 210 ensures continuous improvement in fraud detection capabilities. As new fraud patterns are identified and verified, the system adapts its models and detection strategies, creating an increasingly robust defense against evolving fraud techniques across all levels of sports competition.

Machine learning network 210 implements a comprehensive modeling framework that incorporates multiple sport-specific Al architectures. For football analysis, the system may integrate geometric deep learning models similar to TacticAI, enabling sophisticated analysis of player positioning and strategic events like corner kicks. The preprocessor standardizes inputs from the database 100 and PH1DB 120 to support various sport-specific Al subsystems, each utilizing convolutional neural networks and transfer learning techniques to predict player performance based on historical data.

In one embodiment, machine learning network 210 incorporates advanced modeling techniques that significantly enhance the platform's predictive capabilities. The system implements dynamic game telemetry integration through the data preprocessor 200, which contextualizes real-time state information for both individual players and team dynamics. This telemetry data feeds into sport-specific generative Al such as 400, 410, and 420 that may utilize Super UCT algorithms for sophisticated state transition modeling. The implementation enables dynamic lookback analysis, current state estimation, and look ahead forecasting through variable branching factors that adapt to ongoing gameplay situations.

An analytics subsystem 1270 processes this telemetry data to track object movements such as balls or pucks in conjunction with player positioning, potentially leveraging Monte Carlo Tree Search (MCTS) methodologies to explore potential future game states. This component works with event processor 1230 to optimize the depth and breadth of state-space exploration based on game context, such as high-impact situations like penalties or fast breaks. A market monitor 1280 uses these insights to continuously adjust betting odds and risk assessments in real- time.

The platform extends its capabilities to youth and amateur sports through comprehensive data collection and analysis features. Integration subsystem 1250 connects with team management platforms similar to GameChanger and TeamSnap, incorporating structured data from formal video content, broadcasts, and extensive scorekeeping. This data feeds into the database 100, enabling the player profile generator 140 to create detailed assessments across various competition levels, from youth leagues to professional sports.

Compliance monitor 1260 works alongside fraud detection subsystem 110 to distinguish between legitimate exceptional performance and potential unfair advantages. For instance, when analyzing performance patterns similar to those of elite athletes like Shohei Ohtani, the system can differentiate natural talent from suspicious performance enhancement through comprehensive pattern analysis. Talent tracker 160 leverages this capability to generate accurate "star recruit" ratings and rankings, which can aid in balancing betting markets and finding talent with scouting agencies 161.

Fantasy game subsystem 1220 implements event-oriented trading capabilities, allowing users to trade fantasy teams and assets in secondary markets. This functionality is supported by transaction subsystem 1210, which manages the financial aspects of trading fantasy teams, player rights, and betting positions. The system incorporates real-time performance data and off-field factors through betting profile generator 430, enabling dynamic valuation adjustments based on player conduct, criminal behavior, or other relevant events.

The platform's architecture supports risk management through odds generation subsystem 1200, which continuously updates valuations based on both on-field performance, historical analytics, and external factors. This component interfaces with user manager 1240 to facilitate collaborative team ownership and trading, while integration subsystem 1250 ensures connection with various external platforms and data sources. The result is a comprehensive system that not only enables sophisticated betting and trading capabilities but also maintains integrity across all levels of competition through robust monitoring and analysis.

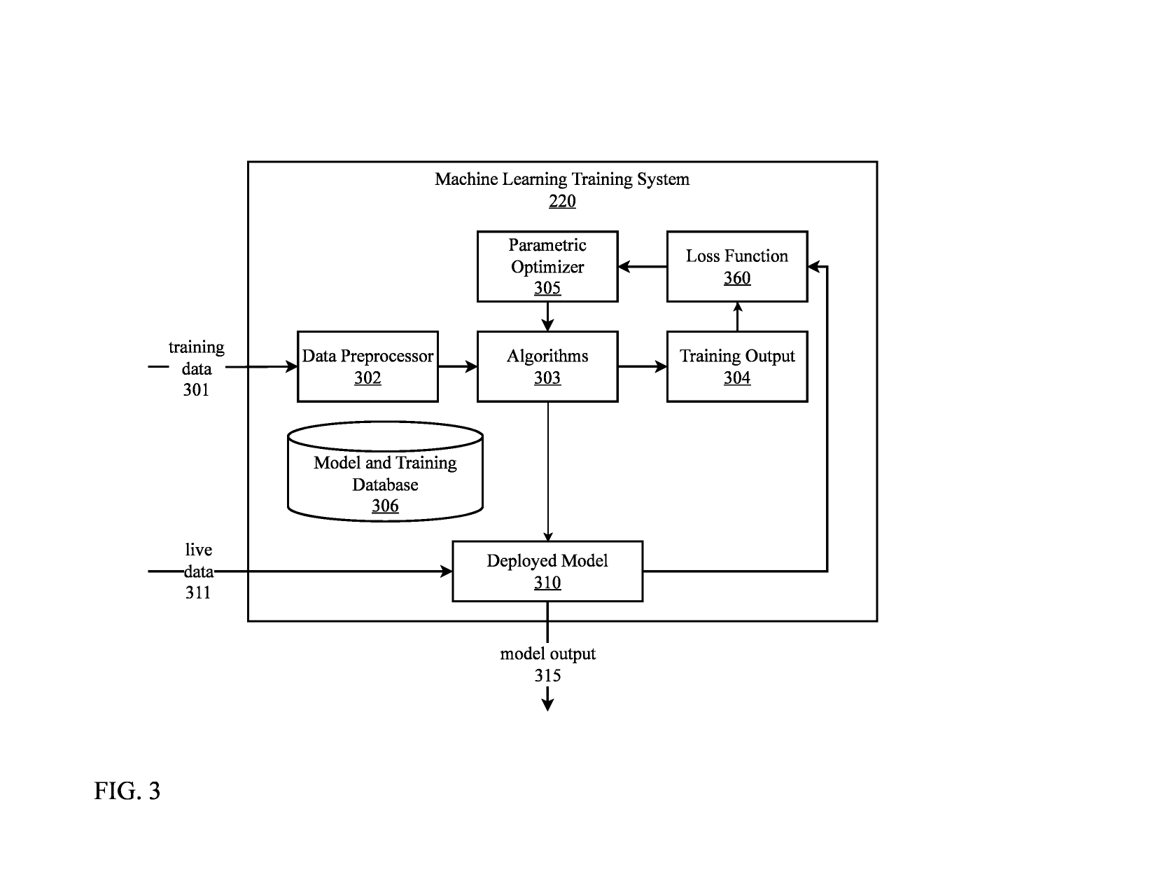

FIG. 3 is a block diagram illustrating a component for a sports betting and gaming platform with integrated AI-based fraud detection, a machine learning training subsystem. According to the embodiment, the machine learning training system 220 may comprise a model training stage comprising a data preprocessor 302, one or more machine and/or deep learning algorithms 303, training output 304, and a parametric optimizer 305, and a model deployment stage comprising a deployed and fully trained model 310 process sports data into a player or user betting profile. The machine learning training system 220 may be used to train and deploy a plurality of deep learning architectures in order to support the services provided by the sports betting and gaming platform with integrated AI-based fraud detection. In one embodiment, machine learning training system 220 may be used to train machine learning network 210. If the machine learning network 210 comprises a plurality of different machine learning architectures, the machine learning training system 220 may train each of the machine learning architectures separately or together as a single system.

At the model training stage, a plurality of training data 301 may be received by the generative Al training system 350. Data preprocessor 302 may receive the input data (e.g., sports betting data, player data, telemetry game data) and perform various data preprocessing tasks on the input data to format the data for further processing. For example, data preprocessing can include, but is not limited to, tasks related to data cleansing, data deduplication, data normalization, data transformation, handling missing values, feature extraction and selection, mismatch handling, and/or the like. Data preprocessor 302 may also be configured to create training dataset, a validation dataset, and a test set from the plurality of input data 301. For example, a training dataset may comprise 80% of the preprocessed input data, the validation set 10%, and the test dataset may comprise the remaining 10% of the data. The preprocessed training dataset may be fed as input into one or more machine and/or deep learning algorithms 303 to train a predictive model for object monitoring and detection.

During model training, training output 304 is produced and used to measure the accuracy and usefulness of the predictive outputs. During this process a parametric optimizer 305 may be used to perform algorithmic tuning between model training iterations. Model parameters and hyperparameters can include, but are not limited to, bias, train-test split ratio, learning rate in optimization algorithms (e.g., gradient descent), choice of optimization algorithm (e.g., gradient descent, stochastic gradient descent, of Adam optimizer, etc.), choice of activation function in a neural network layer (e.g., Sigmoid, ReLu, Tanh, etc.), the choice of cost or loss function the model will use, number of hidden layers in a neural network, number of activation units in each layer, the drop-out rate in a neural network, number of iterations (epochs) in a training the model, number of clusters in a clustering task, kernel or filter size in convolutional layers, pooling size, batch size, the coefficients (or weights) of linear or logistic regression models, cluster centroids, and/or the like. Parameters and hyperparameters may be tuned and then applied to the next round of model training. In this way, the training stage provides a machine learning training loop.

In some implementations, various accuracy metrics may be used by the machine learning training system 220 to evaluate a model's performance. Metrics can include, but are not limited to, word error rate (WER), word information loss, speaker identification accuracy (e.g., single stream with multiple speakers), inverse text normalization and normalization error rate, punctuation accuracy, timestamp accuracy, latency, resource consumption, custom vocabulary, sentence-level sentiment analysis, multiple languages supported, cost-to-performance tradeoff, and personal identifying information/payment card industry redaction, to name a few. In one embodiment, the system may utilize a loss function 360 to measure the system's performance. The loss function 360 compares the training outputs with an expected output and determines how the algorithm needs to be changed in order to improve the quality of the model output. During the training stage, all outputs may be passed through the loss function 360 on a continuous loop until the algorithms 303 are in a position where they can effectively be incorporated into a deployed model 315.

The test dataset can be used to test the accuracy of the model outputs. If the training model is establishing correlations that satisfy a certain criterion such as but not limited to quality of the correlations and amount of restored lost data, then it can be moved to the model deployment stage as a fully trained and deployed model 310 in a production environment making predictions based on live input data 311 (e.g., sports betting data, player data, telemetry game data). Further, model correlations and restorations made by deployed models can be used as feedback and applied to model training in the training stage, wherein the model is continuously learning over time using both training data and live data and predictions. A model and training database 306 is present and configured to store training/test datasets and developed models. Database 306 may also store previous versions of models.

According to some embodiments, the one or more machine and/or deep learning models may comprise any suitable algorithm known to those with skill in the art including, but not limited to: LLMs, generative transformers, transformers, supervised learning algorithms such as: regression (e.g., linear, polynomial, logistic, etc.), decision tree, random forest, k-nearest neighbor, support vector machines, Naive-Bayes algorithm; unsupervised learning algorithms such as clustering algorithms, hidden Markov models, singular value decomposition, and/or the like. Alternatively, or additionally, algorithms 303 may comprise a deep learning algorithm such as neural networks (e.g., recurrent, convolutional, long short-term memory networks, etc.).

In some implementations, the machine learning training system 220 automatically generates standardized model scorecards for each model produced to provide rapid insights into the model and training data, maintain model provenance, and track performance over time. These model scorecards provide insights into model framework(s) used, training data, training data specifications such as chip size, stride, data splits, baseline hyperparameters, and other factors. Model scorecards may be stored in database(s) 306.

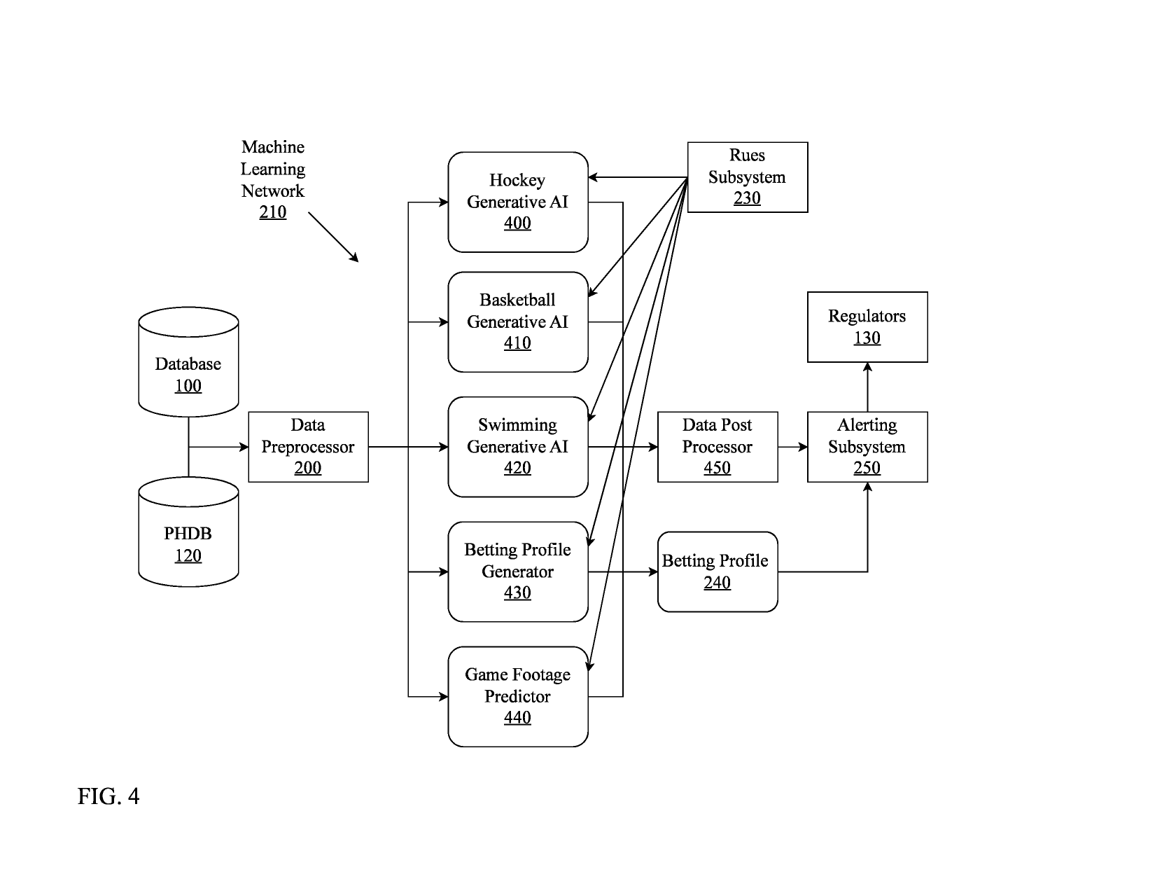

FIG. 4 is a block diagram illustrating a component for a sports betting and gaming platform with integrated AI-based fraud detection, a machine learning network. The network receives standardized data from database 100 and PHDB 120 through a data preprocessor 200 that handles diverse input streams including real-time telemetry, biometric data, and game footage across any sport. While the figure explicitly shows hockey generative Al 400, basketball generative Al 410, and swimming generative Al 420 as examples, the architecture is designed to accommodate Al subsystems for any sport or competitive activity.

Each sport-specific Al component implements architectures optimized for that sport's unique characteristics. For instance, hockey generate Al 400 may employ temporal 1D CNNs and MOT Neural Solvers for player tracking, while basketball generative Al 410 may utilize GANs for analyzing team play patterns, and swimming generative Al 420 may focus on biometric data analysis. Additional sports can be integrated by implementing appropriate Al architectures; for example, football might employ geometric deep learning models like TacticAI, while baseball could use specialized pitch recognition and player form analysis systems.

A betting profile generator 430 aggregates insights from all active sport-specific Al modules to create comprehensive risk assessments. This component is sport-agnostic and can adapt to any new sport added to the system, processing both individual athlete performance data and team dynamics regardless of the sport's specific characteristics.

A game footage predictor 440 works across all integrated sports to generate anticipated performance patterns. This component adapts its prediction methodologies based on the sport being analyzed; from player tracking in team sports to technique analysis in individual competitions. The system may incorporate features like Super UCT algorithms for state transition modeling, allowing for dynamic analysis across any sporting context.

A data post processor 450 standardizes outputs from the various sport-specific Al modules before feeding them to the betting profile 240 and alerting subsystem 250. This ensures consistent fraud detection capabilities regardless of the sport being monitored. The entire system maintains compliance with regulators 130 through the rules subsystem 230, which can adapt to regulatory requirements for any sport or jurisdiction.

The architecture's modular design allows for the integration of new sports and analytical models. Whether analyzing traditional sports, emerging esports, or novel competitive activities, the system can incorporate appropriate Al architectures while maintaining consistent fraud detection capabilities across all platforms. This flexibility ensures the system can evolve with the sports betting landscape while providing comprehensive monitoring across all levels of competition.

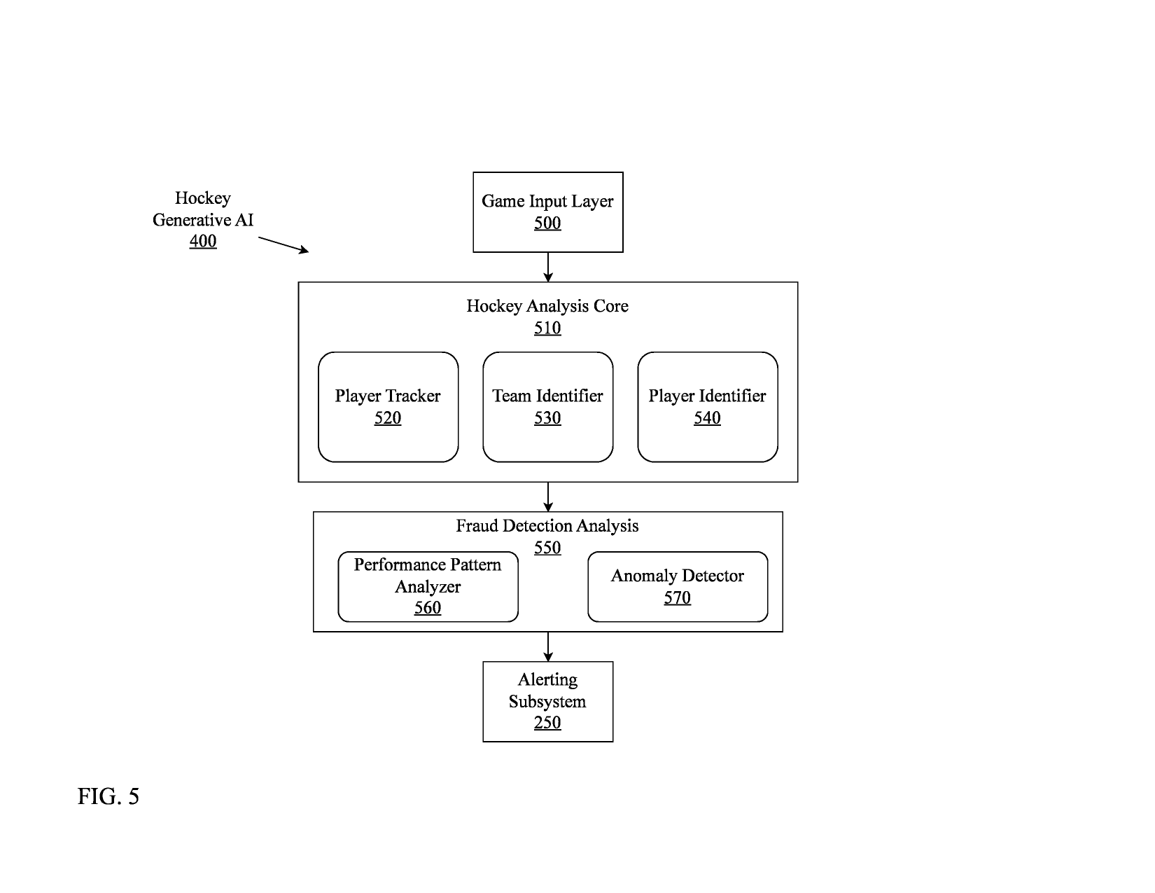

FIG. 5 is a block diagram illustrating a component for a sports betting and gaming platform with integrated AI-based fraud detection, a hockey generative Al. The system begins with a game input layer 500 that ingests broadcast NHL video feeds along with dynamic game telemetry data, including puck movement and player positioning information. A hockey analysis core 510 represents the main processing engine. Within this core, the player tracker 520 may implement a Faster R-CNN with ResNet50 FPN backbone for player detection, followed by a MOT Neural Solver for tracking, achieving a 94.5% Multi-Object Tracking Accuracy MOTA score. This component processes player trajectories as temporal sequences of video frames, handling challenges unique to hockey such as fast-paced, non-linear player movements and frequent camera panning.

In one embodiment, team identifier 530 may utilize a ResNetl8 CNN architecture to classify players into home team, away team, and referee categories. This component groups away team jerseys into a single class while clustering home jerseys based on their colors, enabling effective team identification without requiring retraining for new teams. The player identifier 540 may use a temporal 1D CNN with a ResNetl8 backbone, processing tracklet sequences to achieve jersey number recognition. This component includes post-processing logic to handle cases where jersey numbers are only partially visible or obscured.

Fraud detection analysis layer 550 contains two specialized components. The performance pattern analyzer 560 evaluates player movements and actions using frameworks like deep reinforcement learning to assess player decisions in specific game contexts. This analyzer may leverage optical flow analysis and pose estimation to recognize actions like skating, passing, and shooting. Anomaly detector 570 identifies suspicious patterns by comparing real-time performance data against expected behaviors. For example, it can detect when a player's in-game actions show sudden and unexplained divergence from expected performance patterns. The system utilizes transformer networks and play-by-play data integration to improve the accuracy of these assessments.

Alerting subsystem 250 generates real-time notifications when potential fraud indicators are detected. This component integrates insights from all analytical layers to provide comprehensive risk assessments. For instance, if a player's movement patterns deviate significantly from their historical norms while unusual betting patterns are detected, the system can automatically trigger alerts for further investigation.

This architecture is particularly effective because it combines multiple analytical approaches. The system can simultaneously track player movements, identify teams and individuals, analyze performance patterns, and detect anomalies in real-time. The integration of these capabilities enables sophisticated fraud detection that considers both obvious and subtle indicators of potential misconduct, while maintaining high accuracy rates across all analytical tasks.

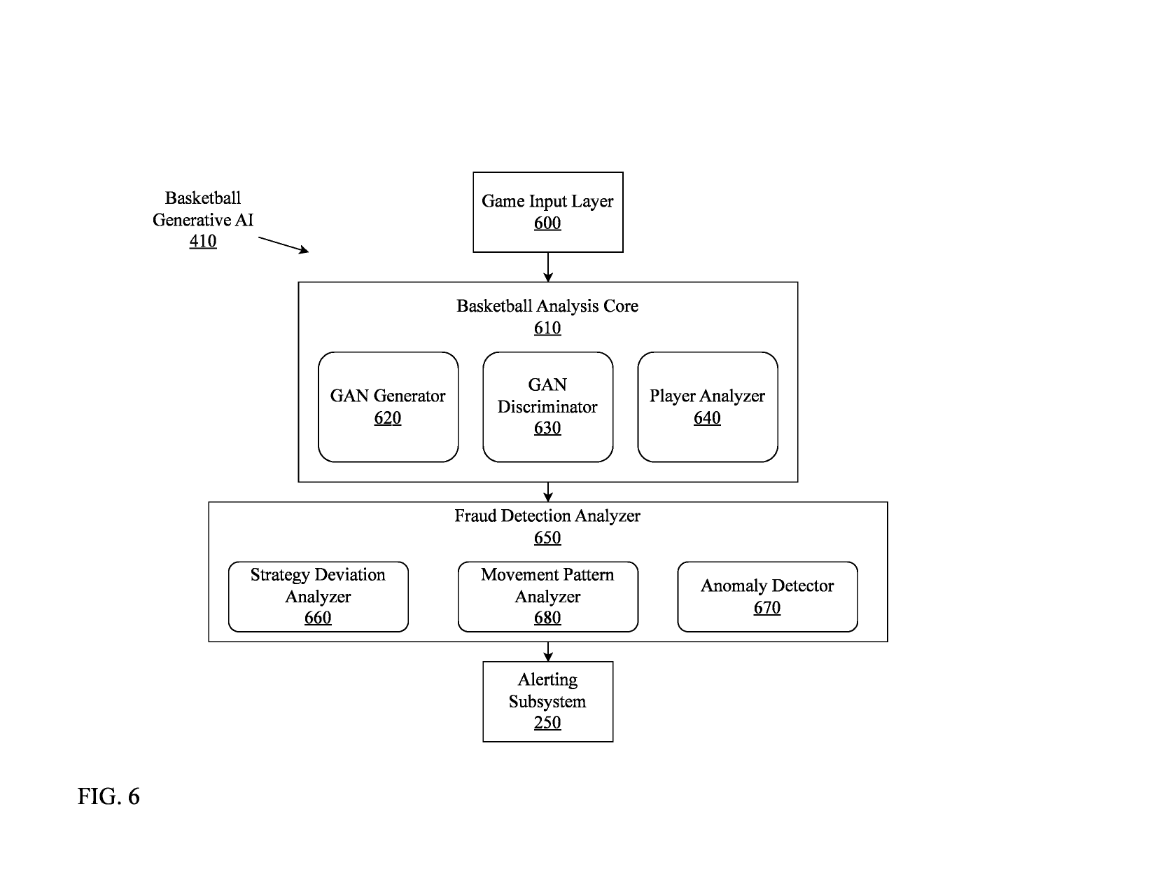

FIG. 6 is a block diagram illustrating a component for a sports betting and gaming platform with integrated AI-based fraud detection, a basketball generative Al. Basketball generative Al 410, may employ generative adversarial networks GANs and specialized analysis components for basketball-specific fraud detection. A game input layer 600 processes basketball game footage along with dynamic telemetry data, including player positions, ball movement, and game event data from platforms like DraftKings and FanDuel where individual player performance metrics are used for betting outcomes.

A basketball analysis core 610 contains GAN components that work in tandem to analyze and validate gameplay patterns. A GAN generator 620 synthesizes realistic basketball gameplay scenarios based on strategy sketches, creating diverse simulations that account for both offensive and defensive strategies. GAN discriminator 630 validates these generated plays against real gameplay patterns, helping to establish a baseline for normal player and team behavior. This GAN architecture, as implemented in BasketballFlow, enables the system to predict player behavior under various in-game conditions and detect anomalous behaviors that might indicate fraudulent activity.

A player analyzer 640 evaluates individual performance metrics and behavior patterns. This component may integrate with SMOGS Social Network Metrics of Game Success to model passing interactions between players as a dynamic network, helping identify patterns that deviate from established norms. For example, sudden changes in a player's passing frequency or quality could signal intentional underperformance.

Fraud detection analyzer 650 contains multiple specialized components. Strategy deviation analyzer 660 monitors how closely actual gameplay adheres to expected strategic patterns, while anomaly detector 670 identifies suspicious deviations in performance. The movement pattern analyzer 680 employs diffusion probabilistic models, similar to PlayBest's approach, to track and analyze player trajectories and decision-making patterns during games.

These components feed into alerting subsystem 250 which generates real-time notifications when potential fraud indicators are detected. For instance, if a player's movement patterns or passing decisions unexpectedly deviate from their established norms while unusual betting patterns emerge, the system can automatically trigger alerts for further investigation.

This basketball-specific architecture is particularly effective because it combines generative modeling with network analysis and pattern recognition. The GAN-based approach allows the system to generate and validate complex gameplay scenarios, while the network analysis components track subtle interactions between players that might indicate collusion or intentional underperformance. The integration of these capabilities enables sophisticated fraud detection that considers both obvious and subtle indicators of potential misconduct in basketball- specific contexts.

The system's ability to analyze both individual and team dynamics makes it especially valuable for monitoring betting activities in basketball, where both player-specific performance metrics and team-level outcomes are common betting targets. This comprehensive approach ensures that suspicious patterns can be detected whether they manifest in individual player statistics, team performance, or specific in-game events.

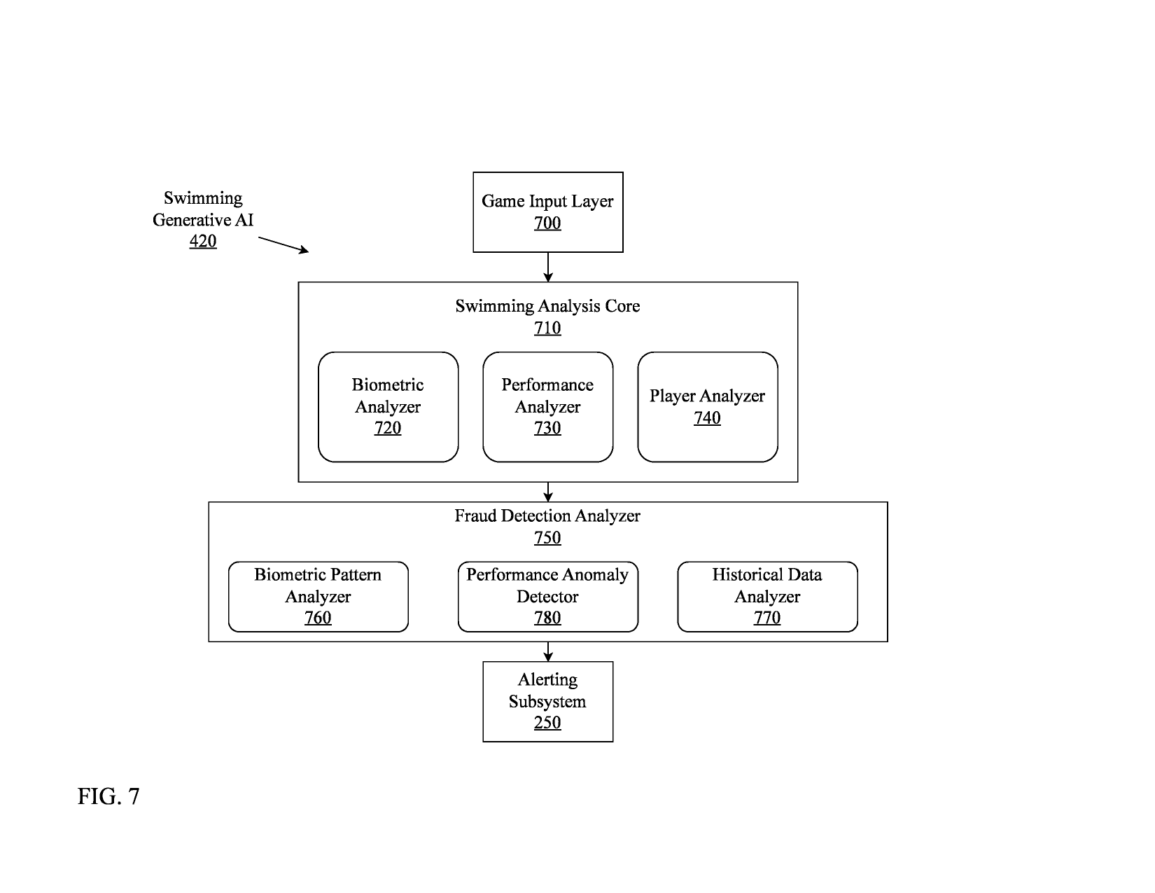

FIG. 7 is a block diagram illustrating a component for a sports betting and gaming platform with integrated AI-based fraud detection, a swimming generative Al. A game input layer 700 captures diverse data streams including but not limited to biometric measurements such as heart rate and stroke count, race timing data, and environmental factors like pool depth and water dynamics that can significantly impact performance.

A swimming analysis core 710 contains components tailored to swimming's unique characteristics as an individual sport. Biometric analyzer 720 processes real-time physiological data from athletes, including heart rate variability, stroke count, and energy expenditure patterns. This component aids in establishing baseline performance metrics and detecting unusual physical exertion patterns that might indicate deliberate underperformance.

A performance analyzer 730 focuses on technical aspects such as lap times, split comparisons, and stroke mechanics. This component can factor in facility-specific variables; for example, how pool depth affects water dynamics and creates turbulence that impacts swimmer speed. Player analyzer 740 builds comprehensive profiles of individual swimmers, incorporating their historical performance data, training patterns, and competition results. Network analysis of player interactions through graph based modeling provides deeper insights into the ranking of players and teams, especially within competitive tournaments. This helps generate more accurate metrics such as "star recruit" ratings which are helpful for betting, NIL rights, and scouting efforts.

A fraud detection analyzer 750 employs multiple specialized components to identify potential fraud. The biometric pattern analyzer 760 monitors for physiological inconsistencies that might indicate deliberate underperformance, such as unexpected changes in stroke rate or energy output that don't align with an athlete's typical race strategy. Historical data analyzer 770 compares current performance against an athlete's historical patterns while accounting for variables like venue familiarity and environmental conditions.

Performance anomaly detector 780 identifies suspicious deviations in race execution. For example, if a swimmer who typically maintains consistent split times suddenly shows irregular pacing while unusual betting patterns emerge, this component would flag the anomaly. These components feed into alerting subsystem 250, which generates real-time notifications when potential fraud indicators are detected.

This swimming-specific architecture is particularly effective because it integrates biometric monitoring with performance analysis in a sport where individual metrics are paramount. The system accounts for both athlete-specific factors like physical condition and race strategy and environmental variables such as pool characteristics that can legitimately affect performance. This comprehensive approach helps distinguish between natural performance variations and suspicious patterns that might indicate betting-related fraud.

The architecture is especially valuable in swimming because of the sport's emphasis on individual performance metrics. Unlike team sports, where complex player interactions can mask irregular behavior, swimming provides clear, measurable performance indicators. The system's ability to correlate biometric data with race outcomes while accounting for facility-specific factors enables highly accurate fraud detection in swimming events, where subtle changes in performance can have significant betting implications.

The system's understanding of facility-specific factors allows it to properly contextualize performance variations. For instance, a swimmer who appears to be underperforming in a shallow pool might be legitimately affected by increased turbulence rather than engaging in fraudulent behavior. This nuanced analysis helps reduce false positives while maintaining high sensitivity to genuine fraud indicators.

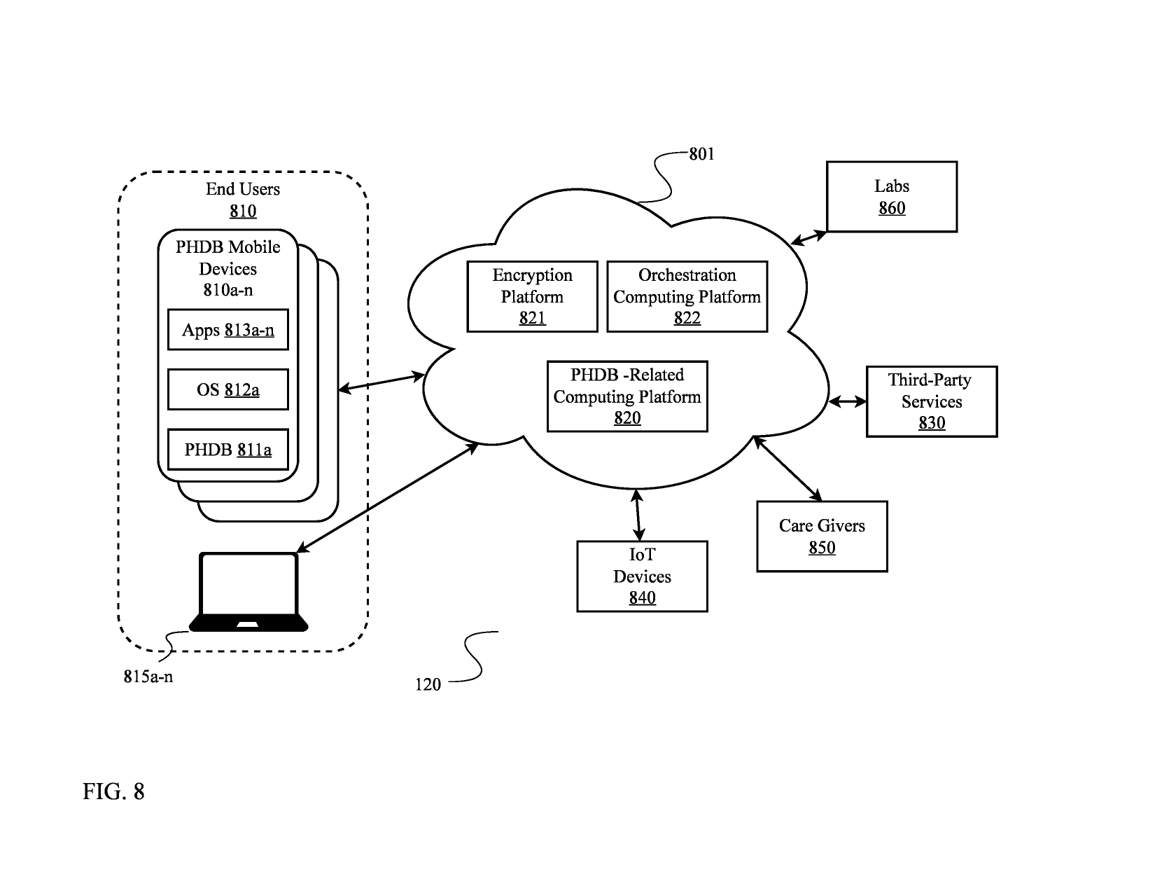

FIG. 8 is a high-level architecture diagram of an exemplary system for using personal health databases (PHDBs), according to an aspect of the invention. As shown in FIG. 8, system 800 offers accessibility to a variety of entities including end users 810, Internet of Things (IoT) devices 840, Care Givers 850, Third-Party Services 830, and Labs 860 by connecting to various cloud-based 801 platforms (e.g., systems, subsystems, and/or services) via a suitable communication network such as the Internet. End Users 810 have flexibility, choosing to engage in cloud-based processing through either their Personal Computers 815a-n which may connect to the cloud-based platforms 801 via a browser-based website or web application, or PHDB- enabled Mobile Devices 810a-n (e.g., smart phone, tablet, smart wearable clothing or glasses, headsets etc.). These mobile devices may comprise a PHDB 811a, an operating system (OS) 812a, and various applications (Apps) 813a-n, creating a comprehensive environment for users to manage and interact with their health and preference data. The ability to have authorized disclosure rules and suggestions or delegate sharing and visibility for personal health records or conditions can also vastly simplify medical procedures and improve outcomes for patients. Current systems force patients into cumbersome manual and often paper disclosure certifications (e.g., outpatient surgery procedure) but could instead be configured to send appropriate status and visibility (even for physical visitation rights in hospital) data to family and friends. This can also better enable post-operative and non-medical facility care by enabling family and friend and personal uploads to the PHDB of photos, interactions, observations, sensor data which can be made available to PHDB processes or to medical staff supporting outcomes.

A user of the system may collect various personal consumption, environment, activity, and other health-related data from a plurality of sources and store the data in their personal health database. Personal health-related data can include genetic information and medical information associated with the user, as well as other types of biometric, behavioral, and/or physiological information. Personal health-related data may be obtained from various sources including, but not limited to, labs 860, third-party services 830, care givers 850, and IoT devices 840. For example, genetic information may be obtained from a lab 860 that conducts genetic carrier screening (e.g., autosomal dominant, autosomal recessive, X-linked dominant, X-linked recessive, mitochondrial, etc.) for a user. Ongoing urine data may be fed from Withings new urine sensor kit, body scan data from an at home body scanner/scale, temperature data from thermal cameras or thermometers, sleep data from smart mattress covers, snoring and sleep quality and sleep apnea indicators from wearable microphones along with heart rate and blood oxygen levels, et cetera. Best practices for individuals or couples wishing to improve personal health outcomes or shared goals such as having children now can include genetic indicator monitoring (e.g. for new papers and research) as well as lived experiences and exposures that may enhance or reduce their risk of adverse health outcomes.

Genetic testing can play a significant role in medical treatment. Some common types of genetic tests that can produce genetic information that can be stored in an individual's PHDB can include diagnostic testing, carrier testing, prenatal testing, newborn screening, pharmacogenetic testing, predictive and presymptomatic testing, forensic testing, and research genetic testing. Diagnostic testing is used to identify or rule out a specific genetic or chromosomal condition. It is done when there is a suspicion based on symptoms or family history. Carrier testing is used to determine if a person carries a gene for a genetic disorder. This type of testing is often done in people with a family history of genetic disorder or in specific ethnic groups with a higher risk. Prenatal testing is conducted during pregnancy to detect genetic abnormalities in the fetus. Examples include amniocentesis, chorionic villus sampling (CVS), and non-invasive prenatal testing (NIPT). Newborn screening involves a series of tests performed on newborns to detect certain genetic disorders early, allowing for early intervention and treatment. Pharmacogenetic testing analyzes how an individual's genes affect their response to certain medications. This information can help personalize medication dosages and selection. Predictive and presymptomatic testing is used to identify genetic mutations associated with conditions data develop later in life, such as certain types of cancer. Presymptomatic testing is done in individuals who do not yet have symptoms but have a family history of a genetic disorder. Forensic testing is used for identification purposes, such as in criminal investigations or paternity testing. Research genetic testing is conducted as part of research studies to better understand the roles of genetics in health and disease. These tests can provide valuable information for healthcare providers, care givers, individuals, and prospective mates.

In some implementations, labs 860 may comprise a plurality of types of labs and facilities that could gather genetic, biometric, behavioral, and/or physiological data on a user. Exemplary labs/facilities can include, but are not limited to, research laboratories (e.g., often affiliated with universities or research institutions and conduct studies to gather various types of data), biotechnology companies, healthcare facilities (e.g., hospitals, clinics, and other healthcare facilities may gather data as part of patient care or research studies. This data could include information from medical tests, imaging studies, and patient questionnaires), tech companies (e.g., wearable technology industry), government agencies, and consumer research firms.

According to the embodiment, caregivers 850 may also provide information to PHDB about the individual which they are providing care for. A caregiver, depending on their role and the context of care, may be responsible for a wide range of medical information. Some common types of medical information that a caregiver might know about or be responsible for include, but are not limited to, patient history (e.g., information about past illnesses, surgeries, medications, allergies, and family medical history), current health status (e.g., information about the patient's current health, including any ongoing medical conditions, symptoms, and vital signs such as blood pressure, heart rate, and temperature), medications (e.g., information about the medications the patient is taking, including dosage, frequency, and any special instructions), treatment plans (e.g., information about the patient's treatment plan, including any medications, therapies, or procedures that have been prescribed), progress notes (e.g., notes on the patient's progress, including any changes in their condition, response to treatment, or other relevant information), diagnostic tests (e.g., information about any diagnostic tests that have been performed, such as blood tests, imaging studies, or biopsies, and the results of those tests), care plan (e.g., information about the overall plan of care for the patient, including goals, interventions, and follow-up care), patient education (e.g., information about legal and ethical issues related to the patient's care such as advance directives, consent for treatment, and confidentiality), and coordination care (e.g., information about coordination of care with other healthcare providers, including referrals, consultations, and care transitions). The specific medical information that a caregiver is responsible for and can provide to the PHDB of their patient will vary depending on the setting and scope of their practice, as well as the needs of the patient.

According to the embodiment, cloud-based platforms 801 may integrate with various third-party services 830 to obtain information related to a user's genetics, biometrics, behavior, and/or physiological characteristics. For example, platform 801 may obtain an electronic health record (EHR), or a subset thereof, associated with the user for inclusion in the user's PHDB.

Additionally, a PHDB mobile device 810a-n may comprise a plurality of sensors which may be used to monitor and capture various biometric, behavioral, and/or physiological data associated with the owner (end user) of the PHDB mobile device. Captured sensors data may be stored in PHDB 811a either in raw data form, or in a format suitable for storage after one or more data processing operations (e.g., transformation, normalization, etc.) has been performed on the sensor data. In some embodiments, a purpose-built software application 813a-n configured to collect, process, and store various sensor data (e.g., biometric, behavioral, physiological, etc.) obtained by sensors embedded into or otherwise integrated with PHDB mobile devices 810a-n. Some exemplary sensors that may be embedded/integrated with PHDB mobile device can include, but are not limited to, fingerprint sensor, facial recognition sensor, heart rate sensor, accelerometer, gyroscope, continuous glucose monitor (CGM), Global Positioning System (GPS), microphone, camera, light sensor, electromagnetic sensors, barometer, pedometer/step counter, galvanic skin response (GSR) sensor (e.g., measures skin's electrical conductivity, which can vary with emotional arousal, stress, or excitement), temperature sensor, lidar, and infrared sensor. More advanced sensors might include Raman- based real-time analytics, gas chromatography mass spectrometry, liquid chromatography mass spectrometry, capillary electrophoresis mass spectrometry, which may be of particular use in environmental exposure considerations in health conditions and lived gene expression. These sensors can be used individually or in combination to gather a wide range of data about the user's biometric, behavioral, and physiological characteristics, enabling various applications such as health monitoring, fitness tracking, personalized user experiences, and human genome filtering for compatibility, to name a few. It is important to note that when combined with temporal and graph representations of interactions in the individual's life, this can feed into a much more nuanced biological monitoring, modeling and simulation aid available for personal, family, or medical use. Users who gather such data fastidiously may also be of particular interest to researchers in support of uncertainty reduction and isolation of particular genetic linkages to this litany of more comprehensive lived factors commonly excluded from static genomics analysis.

In some embodiments, PHDB 811a may be stored in the memory of PHDB mobile or wearable device 810a. In some embodiments, PHDB 811a may be implemented as an encrypted database wherein the plurality of personal health data stored therein is cryptographically encrypted to protect the personal and sensitive data stored therein.

End users 110 may also engage in edge-based processing referring to computing devices that process data closer to the source of data generation instead of relying solely on a centralized server. Edge devices are situated close to the point where data is generated, such as sensors, cameras, or other Internet of Things devices. The Internet of Things (IoT) 840 devices refer to physical objects embedded with sensors, software, and other technologies that enable them to connect and exchange data over the internet. These devices are part of the broader concept of the Internet of Things, which involves the interconnection of everyday objects to the Internet, allowing them to collect and share data for various purposes. Internet of Things devices find applications in various domains, including smart homes, healthcare, industrial automation, agriculture, transportation, and more. Examples include smart thermostats, wearable health monitors, industrial sensors, and connected vehicles. According to the embodiment, a plurality of IoT devices 840 may be deployed to collect and transmit various types of information related to a user's genetics, biometrics, behavior, and/or physiological characteristics. In some implementations, IoT devices 840 can include a plurality of sensors, devices, systems, and/or the like configured to collect and transmit data to cloud-based platforms 801 for inclusion in the user's PHDB. Some exemplary IoT devices 840 can include fitness trackers, smart scales, smart clothing, smart home devices, genetic testing kits, sleep monitors, health monitoring devices (e.g., devices that measure health parameters such as blood pressure, glucose levels, and oxygen saturation, etc.), and wearable cameras.

To facilitate proactive filtering across multiple platforms during interactions with prospective mates, the cloud 801 integrates an optional encryption platform 821, an orchestration computing platform 822, and a PHDB-related computing platform 820.

According to the embodiment, an optional encryption platform 821 may be configured and deployed to provide strong encryption to protect data from unauthorized access. In addition to using strong encryption algorithms, encryption platform 821 is configured to follow best practices for key management, such as using strong, randomly generated encryption keys, and regularly rotating keys to minimize the risk of unauthorized access. In an embodiment, encryption platform 821 may implement advanced encryption standard (AES) for encrypting the various data stored in PHDB. AES is a symmetric encryption algorithm that is widely used and considered to be very secure. It is often used to encrypt data at rest, such as files stored on PHDB. In an embodiment, encryption platform 821 may utilize RSA which is an asymmetric encryption algorithm commonly used for encrypting data in transit, such as data sent over the Internet. In another embodiment, elliptic curve cryptography (ECC) may be implemented which is an asymmetric encryption algorithm that is known for its efficiency and security. In some embodiments, ECC may be used to encrypt data obtained and transmitted by IoT devices 840 to cloud-based platforms 801. In some implementations, a combination of encryption schemes may be utilized to provide secure data storage and transmission. For example, personal-health data may be encrypted in the cloud using RSA and then sent to an end user mobile device 810a wherein it may be encrypted using AES for storage on PHDB 811a of the mobile device.

In some embodiments, encryption platform 821 may implement homomorphic encryption when processing or otherwise analyzing personal health information. In this way, the system can provide processing of encrypted data without having to decrypt and potentially leak personal information.

An orchestration platform 822 is present and configured to provide automated management, coordination, and execution of complex tasks or workflows. This can involve deploying and managing software applications, provisioning and managing resources, and coordinating interactions between different components of system 800. Orchestration platform 822 may automate the deployment and management of virtual machines, containers, and other resources. This can include tasks such as provisioning servers, configuring networking, and scaling resources up or down based on demand. For example, orchestration platform 822 may define and execute a workflow related to the collection, encryption, and distribution (to the appropriate PHDB) of user health-related information. Of particular importance is the ability of the platform to interact with AI/ML systems to aid in explaining, modeling, extracting models, or generating potential items of interest for consideration by medical experts or users. This is further enhanced by the ability for automated planning and modeling simulation services to consider forward scenario analysis of factors (e.g., what if I stopped eating bacon every morning and walked a minimum of 15,000 steps per day instead of my current activity level). This may also help generate financial models to aid users in their personal decision-making and potentially for insurers or medical professionals in theirs. This is becoming more important in the emerging CRISPR/Cas9 and with the sudden emergence of Ozempic and Zapbound era. This is likely to become more challenging for payers, patients and providers if the expected multireceptor agonists such as LY3437943 (a novel triple agonist peptide at the glucagon receptor (GCGR), glucose-dependent insulinotropic polypeptide receptor (GIPR), and glucagon-like peptide-1 receptor (GLP-1R)), emerge and potentially offer large benefits to at risk populations with severe disease and broad-based disease risk factor reductions. Such financial "what if' scenarios will become important when considering the lifetime value of treatment options and potential payment models and is critical to improving patient outcome and better managing continuity of care for healthier and ultimately cheaper patients.

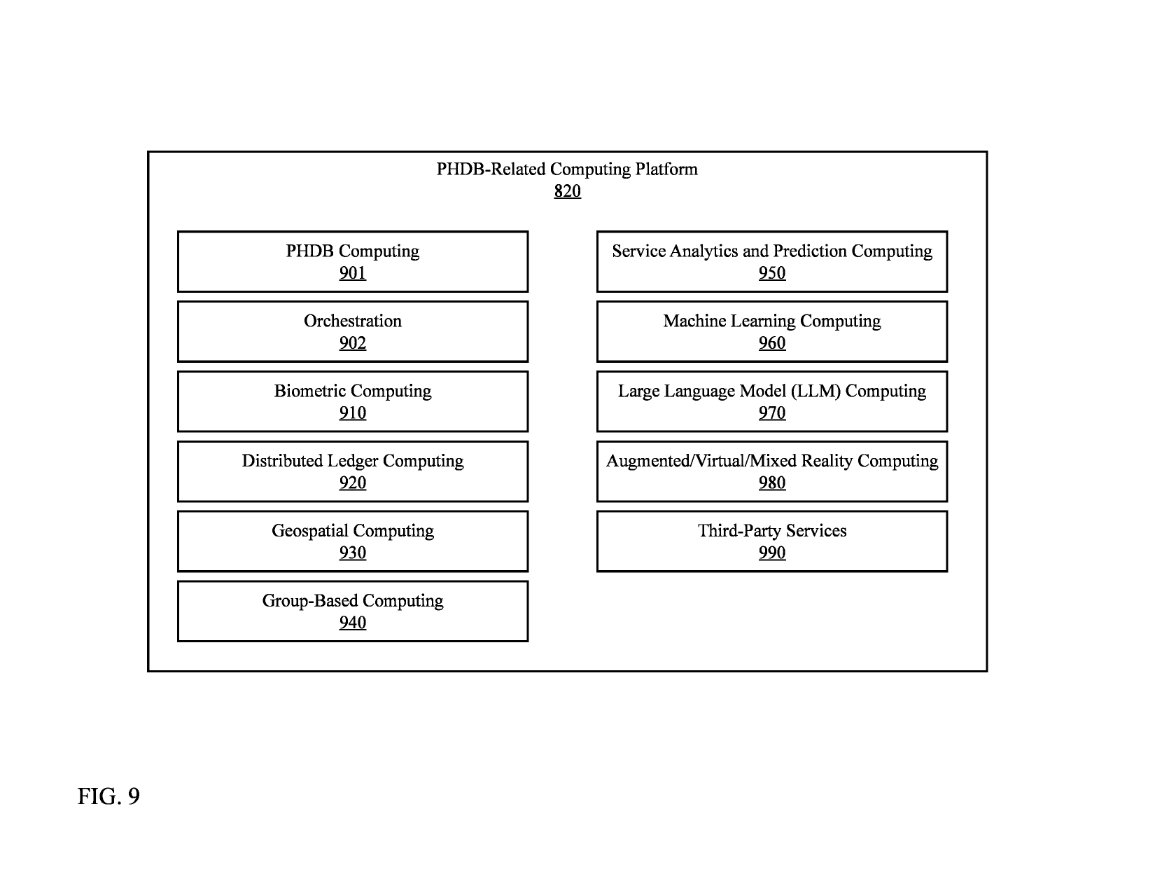

FIG. 9 is a system diagram showing an exemplary arrangement of cloud-based PHDB- related computing, according to an aspect of the invention. According to the aspect, the PHDB computing platform 820 may be implemented as a computing system comprising one or more hardware processors configured for providing the various functionality described herein. According to another aspect, the PHDB computing platform 820 may be implemented as non- transitory, computer-readable storage media having computer-executable instructions embodied thereon and executed by one or more hardware processors configured for providing the various functionality described herein. According to another aspect, the PHDB computing platform 820 may be a computer-implemented method executed by the platform.

According to the embodiment, PHDB-related computing platform 820 comprises various computing components configured to provide computing functionality directed to various categories to support the discrete filtering of two or more human genomes for compatibility using personal health databases. These exemplary computing components may be implemented as servers or services which provide on-demand computing when queried by a system or process. For example, a PHDB-enabled mobile computing device may query geospatial computing 930 to locate and map other PHDB users in the location of mobile device user, and responsive to the query, geospatial computing 930 can send the identified and mapped locations of the other users to the mobile device for display in an application.

PHDB computing 901 is specialized in managing and processing PHDB data, ensuring efficient access and retrieval of diverse health and preference information. Orchestration 302 is responsible for coordinating and managing the flow of data and processes within the system, ensuring seamless interactions between different computing components.

Biometric computing 910 focuses on the analysis and interpretation of biometric data, including fingerprints, face prints, voice prints, and gait data.

Distributed Ledger Computing 920 utilizes distributed ledger technology for secure and transparent record-keeping of sensitive health and preference data, ensuring data integrity and privacy.

Geospatial computing 930 offers ready-to-use demographic datasets and map/imagery layers that allow users to gain immediate context to applications of all types. Group-based computing 940 offers the use of collaboration tools and software that enable multiple users to work together on shared tasks. Service analytics and prediction computing 950 involves leveraging data analytics and predictive modeling techniques to enhance the delivery and optimization of services. Machine learning computing 960 refers to the use of computing systems and algorithms to enable machines to learn and make predictions or decisions based on data.

Large Language Model (LLM) computing 970 refers to the utilization of advanced computational systems to train, deploy, and utilize large language models. These models are typically built using deep learning techniques and have the capability to understand, generate, and process human language at a sophisticated level.

Augmented/Virtual/Mixed Reality computing 980 involves integrating digital information, such as graphics, audio, and other sensory environments, with the user's real-world environment in real-time. Enhances the user's perception of the physical world by overlaying computer-generated content onto it.

Third-Party Services 990 engages external entities to provide additional services, expanding the functionality and capabilities of the PHDB-related computing platform.

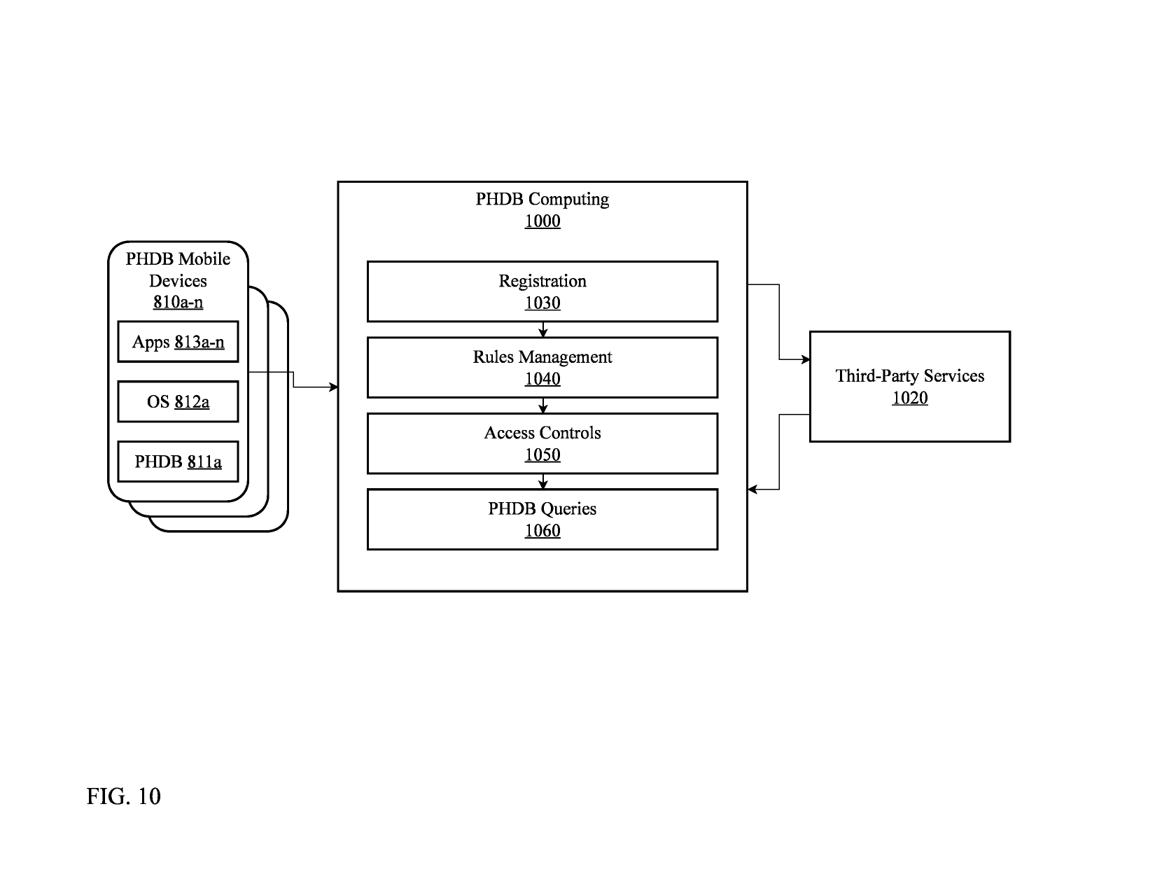

FIG. 10 is a system diagram showing an exemplary arrangement of a PHDB computing server or service with registration controls and third-party service integration, according to an aspect of the invention. The components of PHDB computing 1000 initiate a process that commences at the PHDB mobile device 810a-n, encompassing apps 813a-n, an OS 112a, and PHDB 811a. The process then progresses through the stages of PHDB computing 1000 which acts as an intermediary in the process flow, facilitating seamless interactions between the PHDB mobile device 810a-n and various third-party services 1020. For example, a user of PHDB mobile device 810a may grant permission for social media data related to their likes, check-ins, and subscribed pages data to be uploaded to their PHDB. In this example, PHDB computing 1000 may query a social media server to retrieve the social media data, process the retrieved social media data (e.g., encrypt, transform, format, etc.), and then send the processed data to the PHDB mobile device 810a for storage in PHDB 811a.

Registration 1030 initiates the registration process for PHDB services to users, ensuring proper onboarding and authentication. Registration may store login credentials of a user for various third-party services 1020 to facilitate data exchange. Rules management 1040 manages and enforces rules governing the access and usage of PHDB services, contributing to secure and compliant operations. Rules management 1040 may be aware of or acquire user-defined access rules for their PHDB and apply those rules to adhere to the user's set permissions regarding access to their personal health information. Access Controls 1050 implements controls (e.g., based on user defined rules, or governing rules and regulations such as HIPAA) to regulate access to PHDB services, safeguarding sensitive data and ensuring that users adhere to established rules. PHDB Queries 1060 facilitates queries and requests related to the PHDB, enabling users to retrieve specific information or perform actions within the PHDB ecosystem.

In some implementations, personal health information within the PHDB may be shared with the third-party services 1020. This integration ensures that the PHDB ecosystem can seamlessly interact with and contribute to third-party services.

This exemplary arrangement offers a comprehensive system for managing PHDB services, from user registration 1030, to access controls 1050, and facilitates the sharing of genomic data with external third-party services 1020, thereby expanding the functionality and reach of the PHDB ecosystem.

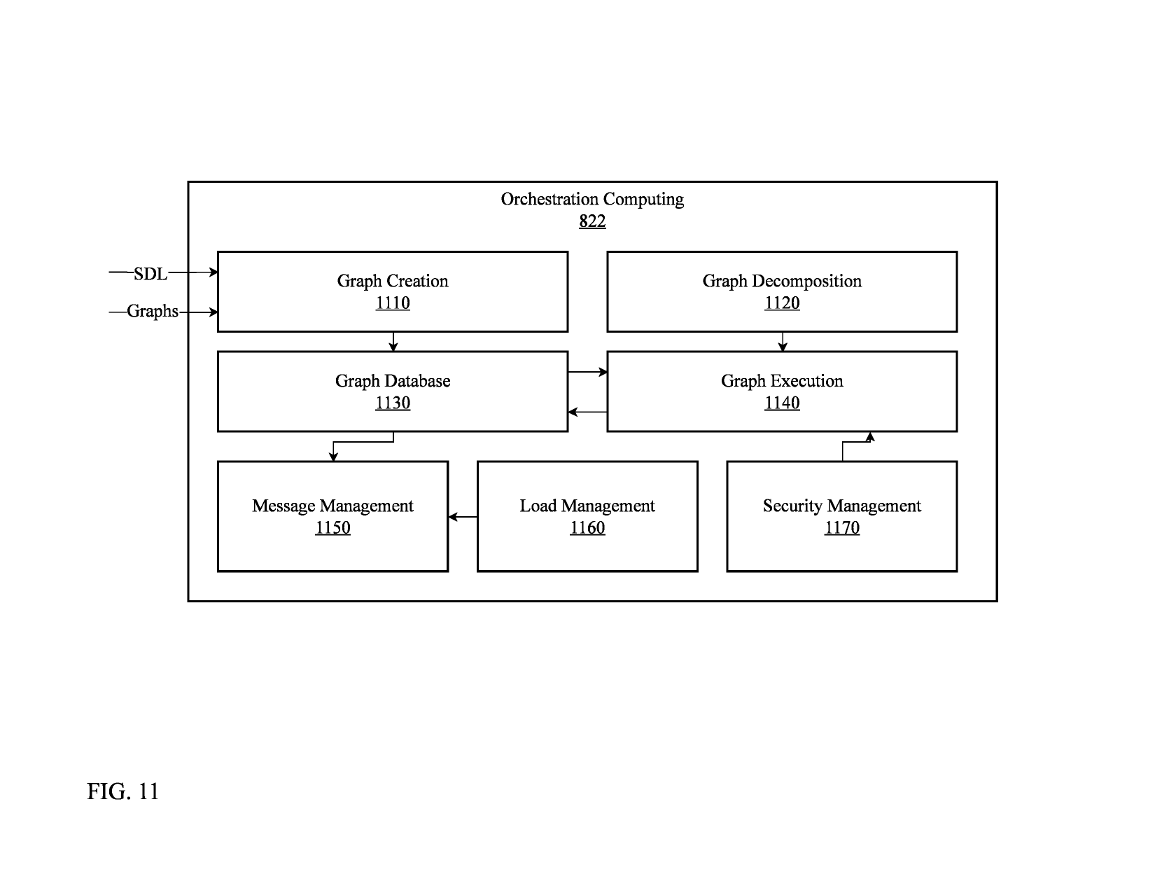

FIG. 11 is a system diagram showing an exemplary arrangement of a PHDB orchestration service according to an aspect of the invention. The orchestration computing 822 initiates a process that involves graph creation 1110 which occurs when a user requests a service, the orchestration service receives the service description and creates a service graph. As shown, graph creation 1110 may receive as input service description language (SDL) data. It is a language used to describe the services, their dependencies, and the workflow of a distributed system. SDL is used to create a formal description of the services and their interactions, which can then be used by orchestration computing 822 to automate the deployment, scaling, and management of the services. Additionally, or alternatively, graph creation 1110 may receive as input a graph to be executed by orchestration computing 822.

The graph creation process results in the establishment of a graph database 1130 wherein created graphs may be stored and retrieved for use. For example, a graph may represent various services that need to act on some data to perform the requested process. Graphs are often used in orchestration computing to represent the relationships between different components or tasks in a system. These graphs, known as orchestration graphs or workflow graphs, can help visualize the dependencies between tasks and the flow of data or control through the system.

Graphs can represent the workflow or sequence of tasks that need to be executed to complete a job or process. Each node in the graph represents a task, and the edges represent the dependencies between tasks. Graph decomposition 1120 is a process where the created graph undergoes decomposition, breaking it down into subgraphs. This step is integral to optimizing service delivery and resource utilization. For example, each subgraph may represent a single service and the data processing required thereof. Graph execution 1140 occurs when the decomposed graph is executed, leading to the activation of subgraphs. This stage ensures that the intended service actions are carried out effectively. The graph database 1130 stores and manages the created graph, serving as a repository for orchestrating service delivery. Message management 1150 is the process where messages are exchanged between subsystems to orchestrate the delivery of messages. Message may comprise data that is to be operated on or can include instructions for processing. During graph execution, security management 1170 ensures the integrity and confidentiality of the controls and graph. This includes implementing measures to protect against unauthorized access or manipulation of genomic data. Load management 1160 oversees the distribution of workloads during graph execution, optimizing system performance and resource allocation. This can involve deploying, scaling, and managing complex applications or services across a distributed environment. Graphs can be used to allocate resources dynamically based on the requirements of different tasks or components. For example, a graph can help determine where to deploy a new instance of a service based on current resource availability and workload.

This orchestration service provides a systematic and efficient approach to coordinating the delivery of services within the PHDB ecosystem. By decomposing and executing service graphs, managing messages, ensuring security, and optimizing load distribution, orchestration computing service 822 enhances the overall efficiency and reliability of service delivery.

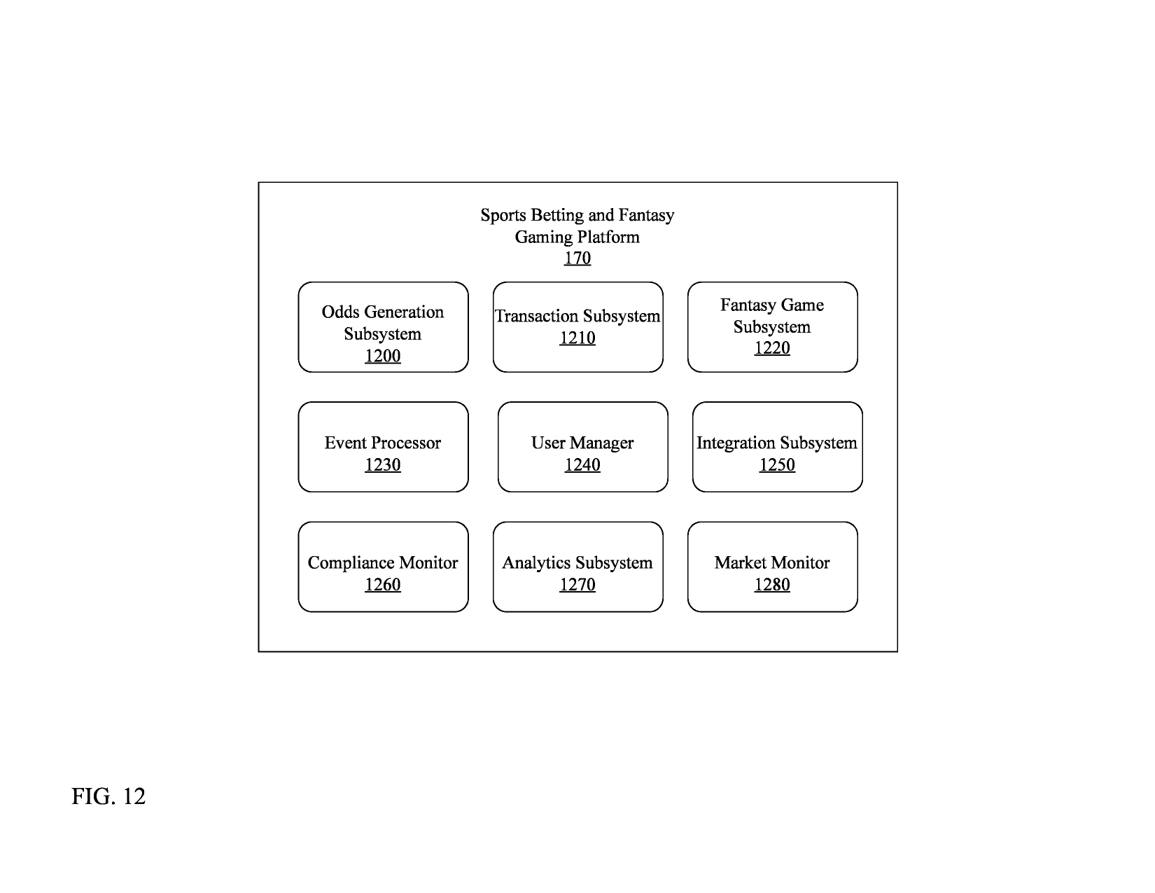

FIG. 12 is a block diagram illustrating a component for a sports betting and gaming platform with integrated AI-based fraud detection, a sports betting and fantasy gaming platform. The internal architecture of sports betting and fantasy gaming platform 170 includes subsystems that work in concert to provide secure, compliant, and engaging betting and gaming experiences. An odds generation subsystem 1200 utilizes Al models to calculate real-time odds across various sports and betting types. This subsystem leverages insights from neural networks and modern portfolio theory to forecast match outcomes, incorporating complex variables such as team/player statistics, historical performance, and current betting patterns. For example, when analyzing basketball games, it can integrate insights from BasketballFlow's GAN simulations to adjust odds based on predicted player behaviors and team strategies.

A transaction subsystem 1210 handles all betting-related financial operations, including bet processing, payment handling, and settlement systems. This subsystem is particularly useful for managing "event-oriented trading" where participants can actively trade fantasy teams or betting positions based on real-time performance data. For instance, if a star player sustains an injury, the system enables team owners to quickly adjust their positions or hedge their bets across multiple markets.

A fantasy game subsystem 1220 manages team creation, roster management, and scoring across various fantasy sports formats. This component implements features that allow fantasy performance to be influenced by real-world athletic achievements, creating dynamic ecosystems where personal performance data can impact fantasy outcomes. For example, a player's actual performance in a local league could earn bonuses or penalties for their fantasy team. Event processor 1230 tracks live sports events and updates platform data in real-time. This subsystem integrates with various data sources, including but not limited to game telemetry, biometric data, and environmental factors, to provide accurate and timely updates that influence both betting odds and fantasy scoring.

A user manager 1240 handles account administration, risk profiling, and user activity monitoring. This component facilitates implementing "know your customer" KYC protocols and preventing fraud through careful monitoring of user betting patterns and account activities. Integration subsystem 1250 manages connections with external platforms and partners, enabling features like real-time updates in gaming platforms such as Madden or FIFA based on actual sports events. This subsystem may also facilitate "participatory gaming experiences" where real- world statistics can affect in-game activities and virtual tournaments.

Compliance monitor 1260 ensures adherence to regulatory requirements across different jurisdictions, implementing real-time checks and reporting mechanisms. This is particularly important given the varying regulatory requirements across different regions and levels of competition, from professional to amateur sports. Analytics subsystem 1270 provides comprehensive performance analysis and trend detection capabilities. This component integrates with various sport-specific Al models to analyze performance patterns and identify potential anomalies that might indicate fraudulent activity.

A market monitor 1280 oversees market dynamics and risk management, tracking betting patterns and market movements to maintain platform stability. This architecture enables features like real-time fantasy team trading, performance-based gaming experiences, and comprehensive fraud detection while maintaining regulatory compliance. The platform's ability to process diverse data streams and adapt to different sports and competition levels makes it particularly effective at providing engaging betting and gaming experiences while protecting against fraudulent activities.

In one embodiment, betting profile generator 430 creates risk assessments by analyzing both individual and team performance metrics. For baseball applications, the system moves beyond simple outcome tracking to analyze the qualitative aspects of player performance, enabling more accurate odds calculations through the odds generation subsystem 1200. This granular analysis is particularly useful for transaction subsystem 1210 and market monitor 1280, which are capable of managing betting risks across all competition levels, from professional to amateur sports.

The plurality of Al architectures that make up fraud detection subsystem 110 implement real-time monitoring capabilities adapted to each sport's specific characteristics. These architectures may interface with analytics subsystem 1270 to process multiple data streams simultaneously, including but not limited to social media content 104, betting history 103, and game footage 105. When suspicious patterns are detected, such as a player's actions diverging from expected performance models or unusual betting patterns emerging, the alerting subsystem 250 generates automated notifications for further investigation.

A compliance monitor 1260 ensures the platform maintains regulatory compliance across different jurisdictions while adapting to sport-specific requirements. This modular approach allows integration subsystem 1250 to customize fraud detection templates based on local gaming commission rules and regional regulations. User manager 1240 implements additional monitoring for lower-level competitions where participants may lack the awareness or maturity of professional athletes, as illustrated by cases like the baseball player who lost $740,000 due to a $99 bet violation.

Fantasy game subsystem 1220 and event processor 1230 work together to provide real- time updates and performance tracking across diverse sports platforms. These components leverage machine learning models and AI-driven analytics to evaluate player performance, detect anomalies, and adjust odds in real-time. Market monitor 1280 oversees these operations, ensuring the platform can adapt to evolving betting patterns and emerging fraud techniques across different sports and competition levels.

In one embodiment, sports betting and fantasy gaming platform 170 may incorporate machine learning techniques across multiple subsystems to optimize betting strategies and decision-making. Odds generation subsystem 1200 may implement deep neural networks that forecast match outcomes by analyzing complex relationships between team and player statistics stored in the database 100. This component may employ modern portfolio theory approaches, including but not limited to the Kelly Criterion and Sharpe Ratio calculations, to balance risk and reward across betting portfolios, with documented success rates in certain sports markets like the English Premier League.

Analytics subsystem 1270 may employ machine learning models for match outcome forecasting, processing historical data to evaluate how player performance, team dynamics, and match-related factors influence results. This analysis feeds into market monitor 1280, which continuously adjusts odds and pricing based on real-time inputs. The system combines these insights with game telemetry data processed by event processor 1230 to maintain accurate, real- time odds calculations across multiple sports and betting markets.

Graph attention networks GATv2 and temporal convolutional layers TCN may be implemented within the machine learning network 210 to model complex player interactions and performance patterns. These neural architectures enable the betting profile generator 430 to create detailed performance forecasts for player-specific betting markets. The integration of temporal analysis allows fraud detection subsystem 110 to identify suspicious patterns in both individual and team performance metrics, while compliance monitor 1260 ensures all betting activities adhere to regulatory requirements.

Transaction subsystem 1210 implements machine learning algorithms specifically designed for multi-bet strategy optimization. This component analyzes betting patterns across multiple games and markets, leveraging wisdom of crowds principles to identify market inefficiencies and optimize pricing mechanisms. The system processes this information through the integration subsystem 1250, which ensures data flow between various platform components and external partners, while the user manager 1240 tracks individual betting patterns and portfolio performance.

This comprehensive AI-driven architecture enables the platform to provide sophisticated betting services while maintaining robust fraud detection capabilities. The fantasy game subsystem 1220 leverages these same machine learning models to enhance fantasy sports gameplay, creating dynamic scoring systems that reflect real-world performance predictions. Through the continuous processing of game data, social media content 104, and betting history 103, the platform maintains accurate odds while identifying and preventing potentially fraudulent activities across all levels of competition.

### Detailed Description of Exemplary Aspects

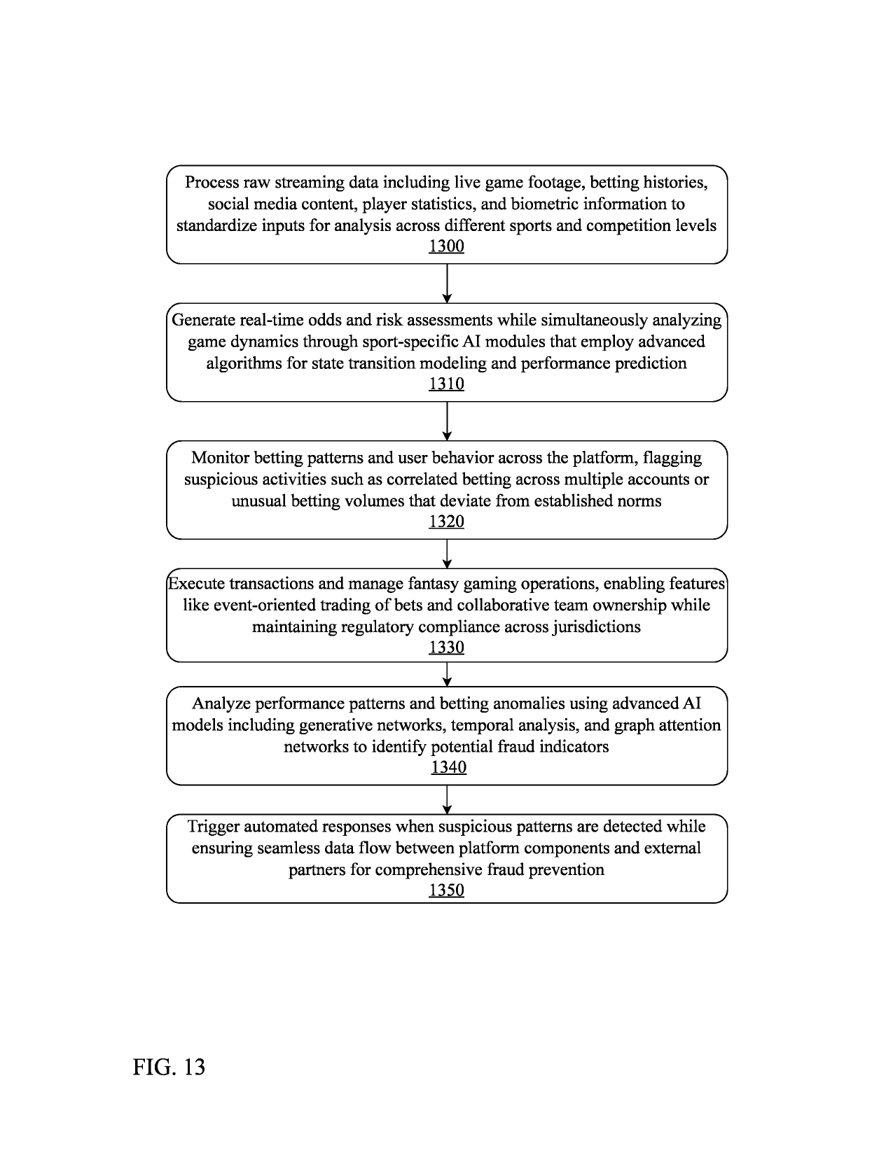

FIG. 13 is a flow diagram illustrating an exemplary method for a sports betting and gaming platform with integrated AI-based fraud detection. In a first step 1300, the system processes multiple data streams through the data preprocessor including real-time game footage, betting histories, social media content, and player data. This step standardizes diverse inputs ranging from biometric data to environmental factors such as facility conditions, creating normalized data formats that can be analyzed across different sports and competition levels, from youth leagues to professional sports.

In a step 1310, an odds generation subsystem produces real-time betting odds while sport-specific generative Al modules analyze game dynamics. This step may employ Super UCT algorithms for state transition modeling, enabling dynamic lookback analysis and lookahead forecasting with variable branching factors that adapt to ongoing gameplay situations, such as high-impact events like penalties or fast breaks.

In a step 1320, a fraud detection subsystem monitors betting patterns while a betting profile generator analyzes user behavior. This step identifies suspicious activities by comparing current betting patterns against established norms, detecting anomalies such as coordinated betting across multiple accounts or unusual betting volumes that could indicate fraudulent activity.

In a step 1330, a transaction subsystem and fantasy game subsystem manage betting operations and fantasy gaming activities. This step enables features like event-oriented trading where participants can trade fantasy teams or betting positions based on real-time performance data, while the compliance monitor (1260) ensures adherence to regulatory requirements across different jurisdictions.

In a step 1340, an analytics subsystem processes performance patterns and betting anomalies using advanced Al models. This step employs various architectures including GANs for gameplay analysis, temporal CNNs for sequence analysis, and graph attention networks (GATv2) to model player interactions and team dynamics, creating comprehensive fraud detection capabilities across multiple sports contexts.

In a step 1350, an alerting subsystem triggers automated responses when suspicious patterns are detected, while an integration subsystem maintains data flow between platform components and external partners. This step ensures rapid response to potential fraud indicators while maintaining system-wide coordination through real-time data sharing and automated alert protocols.

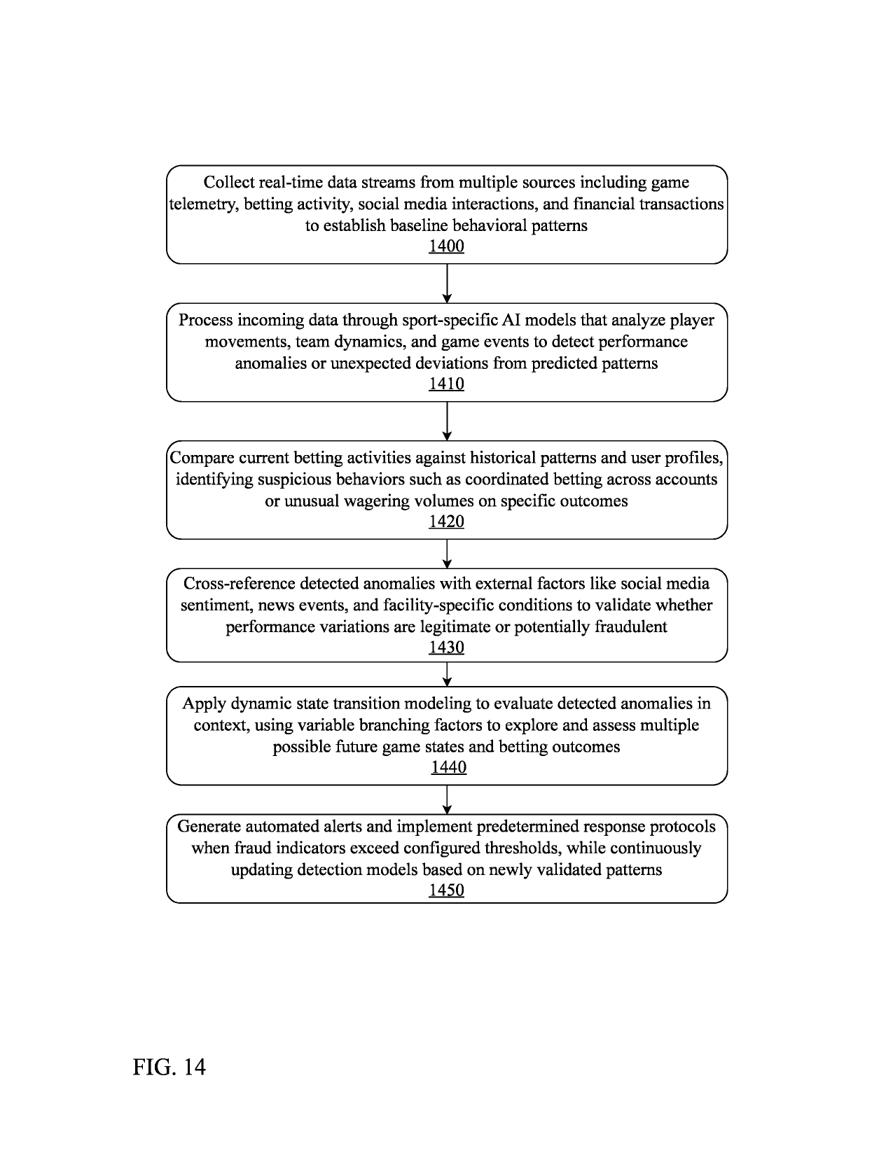

FIG. 14 is a flow diagram illustrating an exemplary method for detecting fraudulent bets made by players or game staff. In a step first 1400, a data preprocessor collects and standardizes real-time data streams from multiple sources feeding into the database. This step aggregates game telemetry such as player tracking data with Multi-Object Tracking Accuracy (MOTA) of 94.5%, betting activity from the betting history, social media interactions, and financial transaction data to establish baseline behavioral patterns for both individual athletes and teams.

In a step 1410, sport-specific generative Al modules analyze the preprocessed data through specialized models. For example, in basketball, the system employs GANs through BasketballFlow to analyze player movements and team dynamics, while in hockey, temporal 1D CNNs achieve accuracy in player identification and movement analysis. This step identifies performance anomalies by comparing real-time behavior against predicted patterns.

In a step 1420, a betting profile generator and analytics subsystem compare current betting activities against historical patterns stored in the database. This step identifies suspicious behaviors such as multiple accounts placing similar high-stakes bets within short timeframes or unusual wagering volumes that deviate from established patterns, as demonstrated in cases like the FanDuel scandal where coordinated betting activities indicated fraudulent behavior.

In a step 1430, fraud detection analyzer components cross-reference detected anomalies with external factors. This step considers facility-specific conditions (such as pool depth in swimming events), social media sentiment analysis, and news events to determine whether performance variations are legitimate or potentially fraudulent, helping distinguish between natural performance fluctuations and suspicious activity. In a step 1440, a machine learning network applies dynamic state transition modeling using Super UCT algorithms to evaluate detected anomalies in context. This step implements variable branching factors that adapt to game situations, enabling the system to explore and assess multiple possible future states while optimizing computational resources based on the significance of different game events

In a step 1450, an alerting subsystem generates automated alerts when fraud indicators exceed configured thresholds. This step implements predetermined response protocols through the rules subsystem, such as suspending betting activity or triggering regulatory investigations, while continuously updating the detection models based on newly validated patterns to improve future fraud detection capabilities.

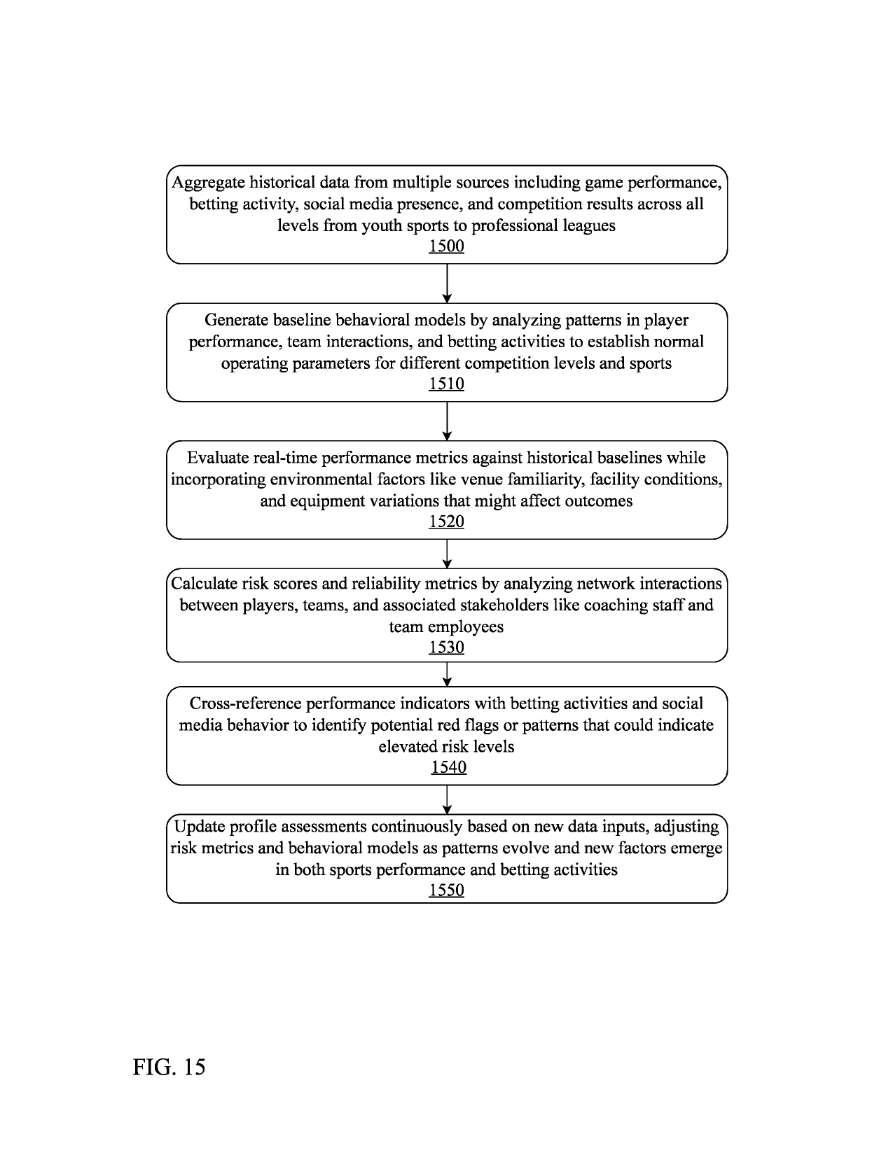

FIG. 15 is a flow diagram illustrating an exemplary method for generating player profiles and betting profiles. In a first step 1500, a database aggregates historical data from multiple sources spanning all competition levels. This step incorporates game performance data from platforms like GameChanger and TeamSnap for youth sports, betting activity records, social media content (104), and professional competition results. A data preprocessor standardizes these inputs to enable comprehensive analysis across different sports and competition levels.

In a step 1510, a player profile generator analyzes patterns to create baseline behavioral models. This step employs sport-specific Al models to understand normal operating parameters - for example, using GAN architectures in basketball to model typical play patterns, or analyzing biometric data patterns in swimming to establish performance baselines. The system accounts for different competition levels, recognizing that patterns in youth sports differ significantly from professional leagues.

In a step 1520, an analytics subsystem evaluates real-time performance metrics against established baselines. This step incorporates environmental factors, such as how pool depth affects swimming performance or how venue familiarity impacts athlete behavior. For instance, the system considers how a swimmer's performance might legitimately vary between pools with different depths due to water dynamics and turbulence effects.

In a step 1530, a machine learning network calculates risk scores by analyzing network interactions using graph attention networks (GATv2) and temporal convolutional layers. This step examines relationships between players, teams, and associated stakeholders, similar to the SMOGS (Social Network Metrics of Game Success) model's analysis of player interactions, while also monitoring non-player staff who might have access to sensitive information or the ability to influence outcomes.

In a step 1540, a fraud detection analyzer components cross-reference performance indicators with betting activities and social media behavior. This step identifies potential red flags, such as unusual performance patterns coinciding with suspicious betting activity, while distinguishing legitimate exceptional performance (like that of Shohei Ohtani) from potential misconduct or unfair advantages.

In a step 1550, a betting profile generator continuously updates profile assessments based on new data inputs. This step dynamically adjusts risk metrics and behavioral models as patterns evolve, incorporating both on-field performance data and off-field factors such as social media activity, public behavior, and equipment changes that might impact performance or betting patterns.

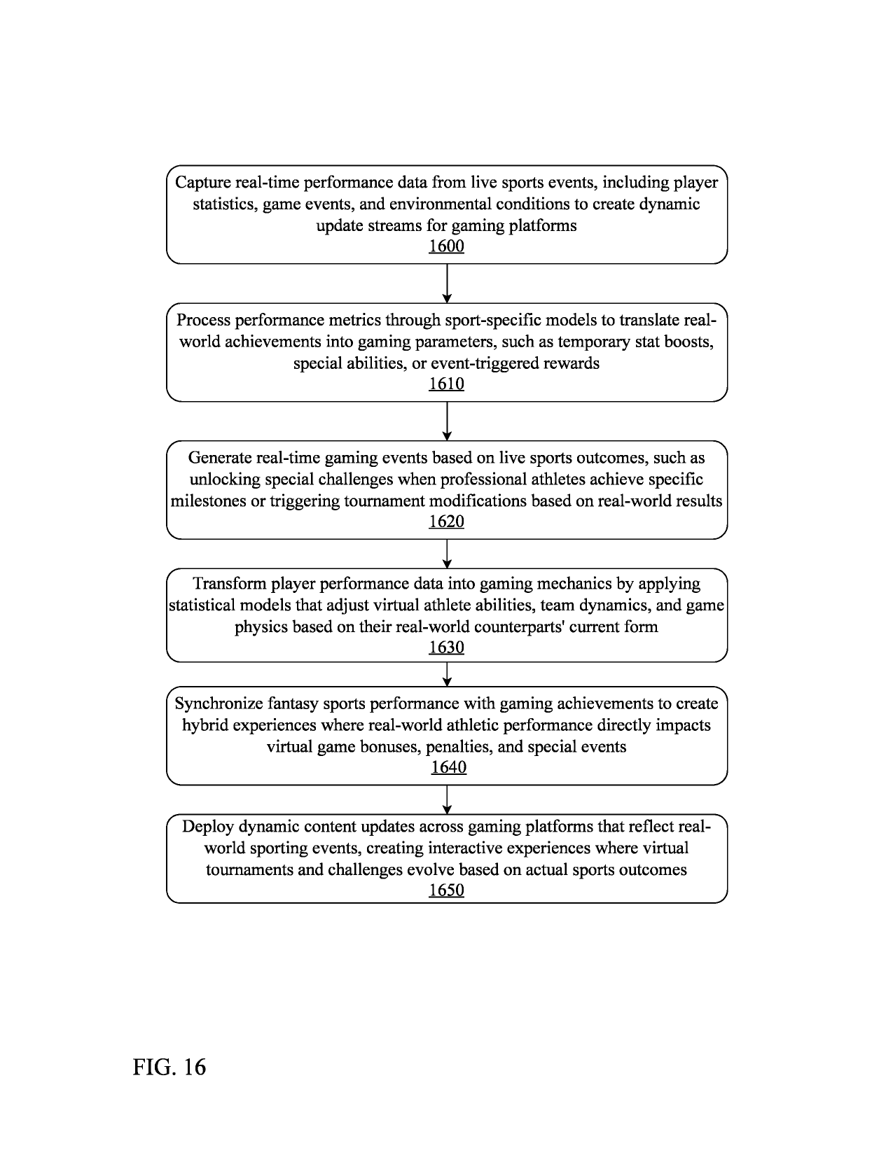

FIG. 16 is a flow diagram illustrating an exemplary method for integrating real time sports data into video games or sports betting platforms. In a step first 1600, the game input layer captures real-time performance data from live sports events. This step collects comprehensive data streams including player statistics, dynamic game telemetry, and environmental conditions such as facility-specific factors. For example, in hockey, the system achieves 94.5% accuracy in player tracking while simultaneously monitoring puck movement and game events.

In a step 1610, a cross-platform subsystem processes performance metrics through sport- specific Al models to translate real-world achievements into gaming parameters. This step employs specialized architectures like BasketballFlow's GAN models to analyze game patterns and convert real-world performance into virtual game modifications, such as temporary ability boosts or special event triggers in platforms like Madden, FIFA, or NBA2K. In a step 1620, an event processor generates real-time gaming events based on live sports outcomes. This step creates dynamic gaming experiences where real-world achievements trigger specific in-game events - for instance, if a basketball player hits a series of three-pointers in a real game, this could unlock special challenges or modify rules in virtual tournaments, creating an interactive link between live sports and gaming platforms.

In a step 1630, an analytics subsystem transforms player performance data into gaming mechanics using advanced statistical models. This step applies sport-specific algorithms to adjust virtual athlete capabilities and team dynamics based on real-world performance. For example, when a professional driver improves their lap times, the system can dynamically recalibrate virtual grid positions or in-game sprint dynamics in F1 simulations. In a step 1640, a fantasy game subsystem synchronizes fantasy sports performance with gaming achievements. This step creates hybrid experiences where real-world athletic performance directly influences virtual game outcomes. For instance, a player's actual performance in local leagues could earn bonuses or penalties for their fantasy team, while their fantasy team's success could unlock special features or challenges in connected gaming platforms.

In a step 1650, an integration subsystem deploys dynamic content updates across gaming platforms. This step ensures that virtual tournaments and challenges evolve based on actual sports outcomes, creating immersive experiences that blend real-world sports with virtual gaming. For example, if a certain number of touchdowns are scored across all NFL matches in one day, players might be rewarded with special in-game items or exclusive virtual events.

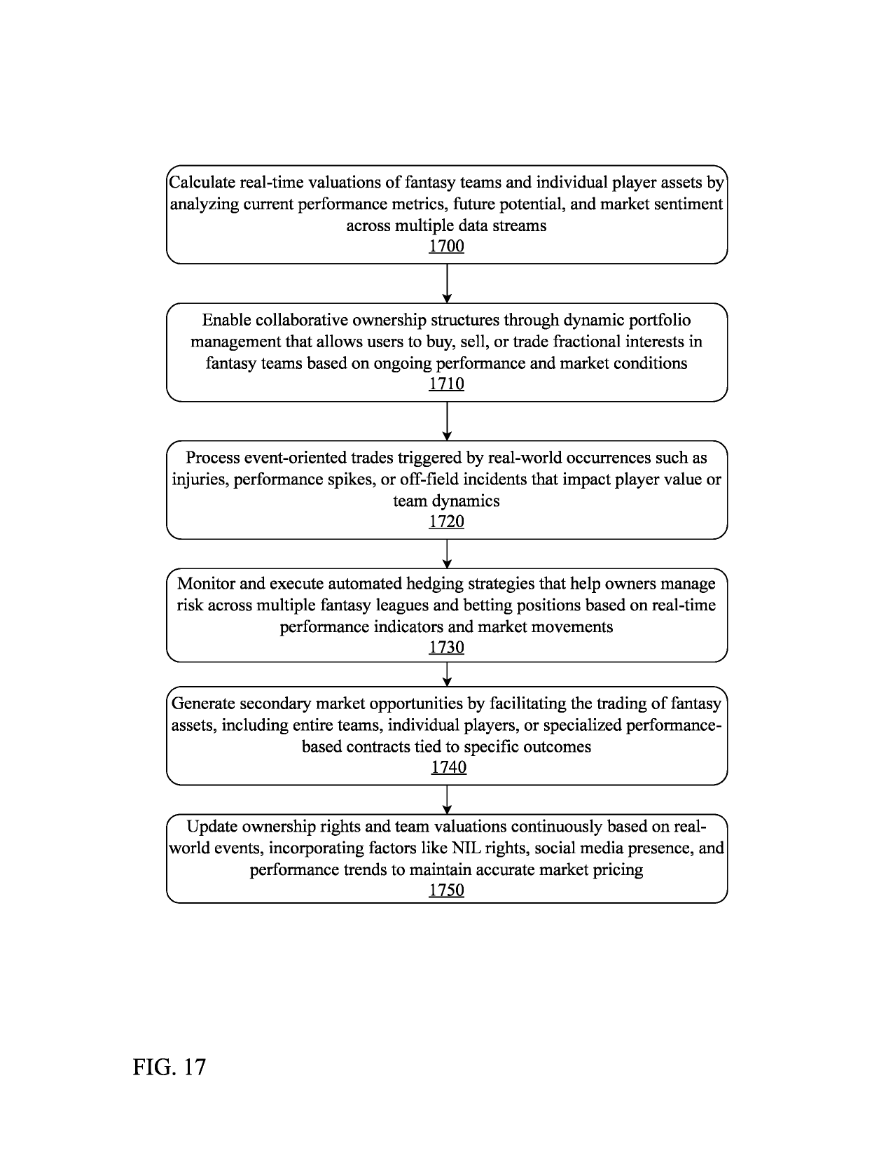

FIG. 17 is a flow diagram illustrating an exemplary method for operating and managing a fantasy team platform. In a first step 1700, an analytics subsystem calculates real-time valuations of fantasy teams and individual player assets. This step aggregates multiple data streams including performance metrics, social media content, and betting patterns to establish dynamic asset values. For example, the system processes player tracking data, historical performance records, and market sentiment analysis to generate comprehensive valuations that reflect both current performance and future potential.

In a step 1710, a transaction subsystem enables collaborative ownership structures through dynamic portfolio management. This step allows users to trade fractional interests in fantasy teams, similar to equity trading, where value fluctuates based on performance metrics, injuries, team changes, and other factors. For instance, if a star player performs exceptionally well, owners can sell portions of their team at a premium or acquire additional stakes in undervalued assets. In a step 1720, an event processor handles event-oriented trades triggered by real-world occurrences. This step processes trades automatically when specific events occur, such as injuries, performance spikes, or off-field incidents like the FanDuel scandal that can dramatically impact player value. The system's ability to monitor both on-field performance and off-field behavior ensures rapid response to value-changing events.

In a step 1730, a market monitor oversees automated hedging strategies across multiple fantasy leagues and betting positions. This step helps owners manage risk by automatically executing trades based on real-time performance indicators and market movements. For example, if a player shows signs of declining performance, the system can automatically hedge positions across different leagues or markets to minimize potential losses.

In a step 1740, a fantasy game subsystem generates secondary market opportunities for trading fantasy assets. This step facilitates the creation and trading of specialized contracts tied to specific outcomes or performance metrics, similar to NIL rights trading. The system enables owners to trade entire teams, individual players, or performance-based contracts, creating liquid markets for fantasy sports assets. In a step 1750, a betting profile generator continuously updates ownership rights and team valuations based on real-world events. This step incorporates various factors including NIL rights valuations, social media impact, and performance trends to maintain accurate market pricing. For instance, when a rookie athlete outperforms expectations, their fantasy value is automatically adjusted to reflect their rising star potential and increased marketability.

In one embodiment, the platform enables sophisticated event-oriented trading through coordinated operation of multiple subsystems. The fantasy game subsystem 1220 may interface with the transaction subsystem 1210 to facilitate real-time trading of fantasy assets based on events detected by the event processor 1230. For example, when performance spikes are identified through the sport-specific generative Al architectures, market monitor 1280 automatically adjusts asset valuations, enabling owners to execute trades through the platform. The user manager 1240 supports collaborative ownership structures where multiple users can pool resources to acquire high-value assets, with compliance monitor 1260 ensuring all transactions adhere to regulatory requirements across jurisdictions.

The odds generation subsystem 1200 continuously updates valuations based on real-time events processed through analytics subsystem 1270, while betting profile generator 430 tracks trading patterns to detect potential market manipulation. When significant events occur - such as player injuries, performance anomalies, or off-field incidents; the system enables rapid portfolio adjustment through automated hedging mechanisms. For instance, if the player profile generator 140 identifies a potential injury risk through PHDB 120 data, or if social media content 104 indicates emerging reputational risks, the platform automatically flags these risks to asset holders and suggests hedging strategies across different markets and leagues. The system's integration subsystem 1250 ensures execution of trades across multiple platforms while maintaining transaction records for regulatory compliance through rules subsystem 230, effectively creating a regulated secondary market for fantasy sports assets that operates similarly to traditional equity trading platforms.

In another embodiment, the platform provides sophisticated risk management capabilities for NIL contracts and associated assets through comprehensive monitoring and automated trading mechanisms. Analytics subsystem 1270 processes multiple data streams to assess athlete value volatility, incorporating game performance data, social media content 104, and biometric information from wearable devices through the data preprocessor 200. Talent tracker 160 monitors athlete performance trajectories while the betting profile generator 430 tracks market sentiment and valuation trends. When the system detects value-impacting events; whether performance-related, health-related through PH1DB 120, or conduct-related through social media monitoring; market monitor 1280 triggers automatic valuation adjustments, enabling stakeholders to execute responsive trading strategies through the transaction subsystem 1210.

The platform's surveillance capabilities are enhanced through continuous monitoring of multiple data sources. Wearable technology data streams are processed through sport-specific Al architectures to analyze biometric patterns, including stress levels, injury risk indicators, and performance metrics. The machine learning network 210 integrates these insights with social media sentiment analysis and public behavior monitoring to provide real-time valuation updates. For instance, if the system detects concerning biometric patterns or negative social media sentiment through natural language processing, the odds generation subsystem 1200 automatically adjusts asset valuations while alerting subsystem 250 notifies stakeholders of potential risks. The compliance monitor 1260 ensures all surveillance and trading activities adhere to privacy regulations and contractual obligations, while the integration subsystem 1250 facilitates seamless interaction between various data sources, trading platforms, and stakeholder interfaces.

User manager 1240 supports sophisticated trading strategies for NIL rights holders and fantasy sports participants, enabling them to hedge positions across multiple markets based on real-time risk assessments. For high-profile athletes, the system tracks performance metrics, public sentiment, and brand alignment through a comprehensive risk profile maintained by player profile generator 140. When event processor 1230 detects significant changes in any monitored metrics - from declined performance to viral social media incidents; the platform automatically executes predefined hedging strategies while maintaining compliance through rules subsystem 230. This integration of surveillance technology, real-time monitoring, and automated trading creates a dynamic risk management ecosystem for modern sports investments.

In another embodiment, the platform expands to incorporate performance optimization and training technologies through integrated biomechanical analysis and feedback systems. The system processes data from exoskeleton-enhanced training sessions through sport-specific Al architectures, analyzes movement patterns and compares them against ideal biomechanical models stored in database 100. Analytics subsystem 1270 processes real-time motion data to generate corrective feedback, while the machine learning network 210 continuously refines optimal movement patterns based on performance outcomes. For instance, when analyzing a baseball pitcher's throwing mechanics, the system employs temporal CNNs to track movement deviations from established optimal patterns, providing real-time guidance, feedback, or stimulus through connected exoskeleton systems or smart material/soft actuated enhanced wearables to restrict or enable specific movement paths according to kinesiologic models while recording performance data for analysis by talent tracker 160.

The platform integrates data from smart wearable sensors and electro-clothing systems through data preprocessor 200, which standardizes inputs from various biometric sensors and EMS devices. This data feeds into player profile generator 140, which maintains comprehensive performance profiles including biomechanical patterns, muscle activation data, and recovery metrics. PH1DB 120 stores individual athlete health and performance baselines, enabling the system to dynamically adjust training parameters through connected EMS systems. Event processor 1230 monitors real-time biometric data streams, triggering automated adjustments to training protocols based on fatigue indicators, stress levels, or injury risk patterns identified through pattern analysis.

Fantasy game subsystem 1220 leverages this expanded data ecosystem to create innovative crossover experiences between real-world performance and fantasy sports. Real-time performance data processed through the analytics subsystem 1270 directly influences fantasy team valuations managed by the market monitor 1280. Integration subsystem 1250 facilitates data flow between physical training systems, virtual platforms, and fantasy leagues, while transaction subsystem 1210 executes value adjustments based on real-world performance metrics. For example, when wearable sensors detect performance improvements during training sessions, odds generation subsystem 1200 automatically adjusts player valuations across fantasy platforms. These capabilities extend to motorsports applications, where real-time telemetry data can dynamically modify virtual racing parameters and fantasy league standings, creating an immersive connection between physical performance and virtual competition while maintaining regulatory compliance through compliance monitor 1260.

In a further embodiment, the platform extends its capabilities to integrate comprehensive movement optimization and injury prevention across both athletic and workplace environments. The player profile generator 140 creates specialized profiles for both professional athletes and casual users, while analytics subsystem 1270 processes biomechanical data from smart clothing and EMS systems. For injury recovery applications, PHDB 120 maintains detailed health records and recovery metrics, enabling machine learning network 210 to generate optimized training protocols. Sport-specific Al architectures analyze movement patterns against ideal kinematic models, while connected EMS suits and exoskeletons provide real-time corrective feedback through the event processor 1230.

The platform implements risk assessment and insurance modeling capabilities through expanded data integration. Data preprocessor 200 aggregates inputs from workplace wearables, environmental sensors, and machinery instrumentation to create comprehensive risk profiles. Machine learning network 210 analyzes this data to identify potential injury risks and safety violations, while market monitor 1280 adjusts insurance premiums in real-time based on observed behaviors and conditions. For transaction-specific insurance processing, event processor 1230 evaluates individual task risk factors, including worker fatigue levels from biometric sensors and environmental conditions from image analysis systems, enabling dynamic premium adjustments through transaction subsystem 1210.

The system's movement optimization capabilities are enhanced through integration with advanced physics modeling and robotic validation systems. Analytics subsystem 1270 incorporates computational fluid dynamics and structural analysis to simulate complex biomechanical interactions, while sport-specific Al modules develop optimized movement patterns for different activities. For example, when analyzing baseball pitching mechanics, the system processes motion capture data through physics models to identify optimal trajectories that maximize performance while minimizing injury risk. Talent tracker 160 monitors skill development across different age groups and ability levels, while compliance monitor 1260 ensures training protocols adhere to safety standards. Real-time feedback from robotic testing systems helps validate these models, with machine learning training system 220 continuously refining movement patterns based on performance data and health outcomes stored in PHDB 120.

In one embodiment, the system may be configured for event-oriented trading and market dynamics through coordinated operation of multiple subsystems. Fantasy game subsystem 1220 interfaces with transaction subsystem 1210 to enable real-time trading of fantasy assets and betting positions. Event processor 1230 continuously monitors game events, performance metrics, and market conditions, while the analytics subsystem 1270 processes this data to generate dynamic asset valuations. When significant events are detected, such as exceptional performance, injuries, or team changes, market monitor 1280 may automatically adjust asset valuations in real-time. The user manager 1240 facilitates collaborative ownership structures, allowing multiple users to pool resources and acquire fractional interests in high-value assets, while the compliance monitor 1260 ensures all transactions adhere to regulatory requirements across jurisdictions. Through this integrated approach, participants can actively manage their portfolios by buying, selling, or trading fantasy assets based on real-time events and performance metrics, creating a liquid secondary market that operates similarly to traditional equity trading platforms.

In another embodiment, the system may be configured for risk management and NIL contract hedging through sophisticated monitoring and automated trading mechanisms. Analytics subsystem 1270 processes multiple data streams to assess athlete value volatility, incorporating real-time performance data, social media content 104, and market sentiment analysis. Machine learning network 210 employs specialized models to evaluate both on-field performance and off- field factors that might impact NIL value. For example, when analyzing high-profile athletes, the system monitors social media sentiment through natural language processing, tracks performance metrics through sport-specific Al modules such as 400, 410, 420, and evaluates market reactions through market monitor 1280. If potential risks are identified, whether performance-related, conduct-related, or market-related, alerting subsystem 250 notifies stakeholders while transaction subsystem 1210 executes predetermined hedging strategies. Betting profile generator 430 maintains comprehensive risk profiles for athletes, incorporating both quantitative performance metrics and qualitative factors like public perception and brand alignment. When significant events occur that might impact NIL value, such as performance declines or off-field incidents, the system enables stakeholders to adjust their positions through automated trading mechanisms while maintaining regulatory compliance through rules subsystem 230. This creates a dynamic risk management ecosystem where NIL rights holders can actively hedge their positions against various forms of value volatility while capitalizing on positive value movements through real- time market access.

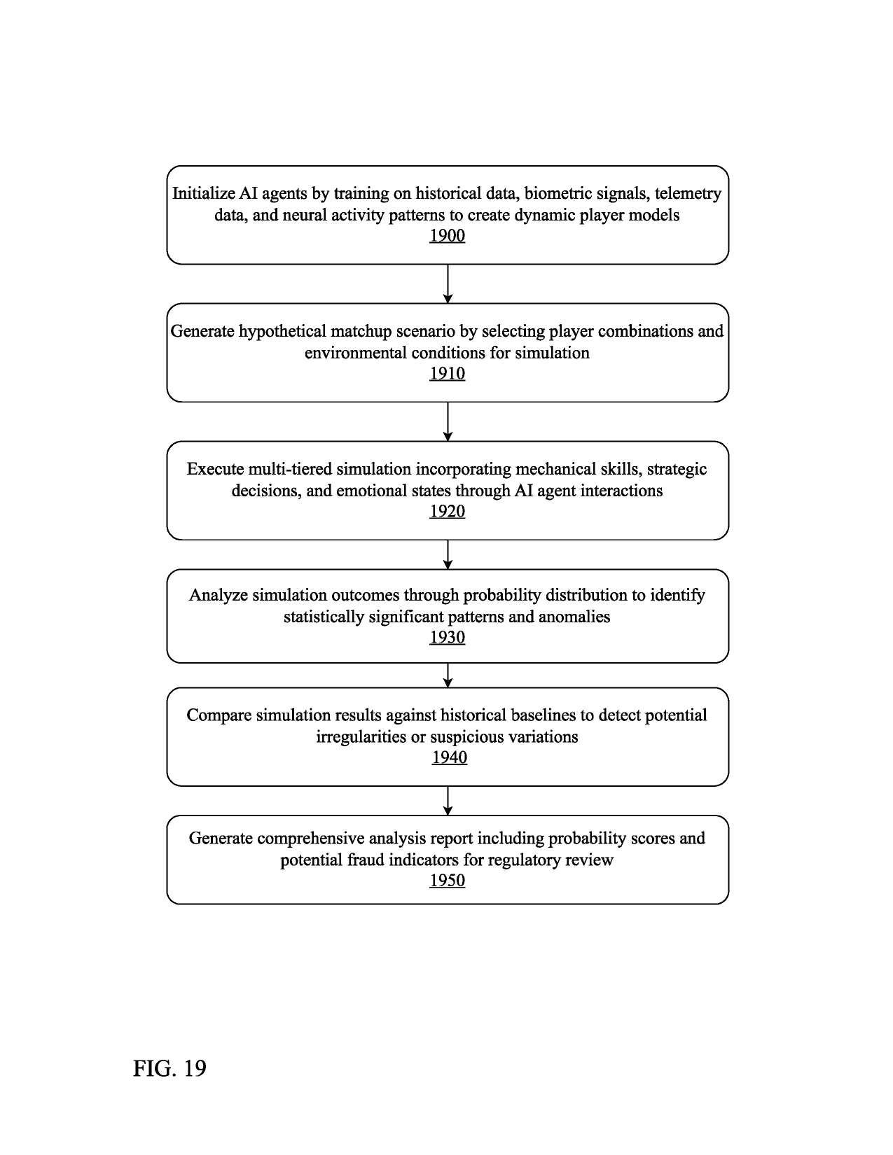

FIG. 19 is a flow diagram illustrating an exemplary method for generating and analyzing hypothetical game scenarios using AI-enabled simulation. The system employs multiple artificial intelligence agents, each trained to emulate specific players or entire teams, to simulate and analyze hypothetical matchups and game scenarios. These agents are created through comprehensive training on historical datasets, biometric signals, and telemetry information, enabling them to exhibit emergent behavior patterns that closely match real-world athletes. Through this architecture, the system can virtually simulate matchups that may never have occurred, repeatedly replay historical scenarios with altered conditions, and generate probability distributions to evaluate the likelihood that certain outcomes were influenced by irregular factors rather than genuine competitive variance. This capability is particularly valuable for fraud detection and integrity assessment, as it allows for detailed analysis of both historical and hypothetical scenarios while providing statistically rigorous evidence of potential manipulation.

In step 1900, the system initializes Al agents through a comprehensive training process that incorporates multiple data streams. This step aggregates historical performance data, real- time biometric signals, telemetry tracking information, and neural activity patterns (such as EEG readings) to create sophisticated behavioral models. Each Al agent is trained using specialized neural networks: one network focuses on mechanical skill execution (like signature movement patterns), another handles strategic decision-making, and a third processes emotional states derived from biometric data. These networks are continuously refined through unsupervised and reinforcement learning processes to ensure the agents exhibit emergent behavior patterns that closely match real-world athletes.

In step 1910, the system generates specific hypothetical matchup scenarios by carefully selecting player combinations and environmental conditions. This step allows for the creation of both realistic matchups that could occur in actual competition and speculative scenarios, such as comparing athletes from different eras or testing how environmental factors might affect performance. The system considers venue characteristics, equipment variations, and other contextual factors that could influence the simulation's outcome.

In step 1920, the system executes multi-tiered simulations through coordinated Al agent interactions. A global orchestrator module manages these interactions, running high-fidelity trials at scale while maintaining temporal coherence. The simulation incorporates mechanical skills (such as a player's signature shooting motion or pitching style), strategic decisions (like play selection or defense positioning), and emotional states inferred from historical biometric data. This creates a comprehensive representation of how players would likely perform in the hypothetical scenario.

In step 1930, the system analyzes simulation outcomes using advanced statistical methods to generate probability distributions. This step processes thousands of simulation iterations to identify statistically significant patterns and anomalies. The system examines various performance metrics, interaction patterns between players, and outcome probabilities to build a comprehensive understanding of what constitutes normal versus suspicious variation in the simulated scenarios.

In step 1940, the system compares simulation results against established historical baselines to detect potential irregularities. This step evaluates whether observed variations in performance or behavior fall within expected parameters or represent statistically significant deviations that warrant further investigation. The comparison takes into account factors such as player fatigue, environmental conditions, and competitive context to ensure accurate assessment of potential anomalies.

In step 1950, the system generates detailed analysis reports containing probability scores and potential fraud indicators. These reports provide regulators with comprehensive insights into the simulation results, including probability distributions of outcomes, identified anomalies, and supporting evidence for any flagged irregularities. The reports include visualizations of key metrics, statistical analyses of performance variations, and specific recommendations for further investigation when suspicious patterns are detected.

This flow enables the system to conduct sophisticated what-if analyses and integrity assessments by leveraging Al agents trained on comprehensive historical and biometric data. The process can be applied to investigate historical events, evaluate hypothetical scenarios, or assess the likelihood of potential future outcomes while maintaining high standards for statistical validity and regulatory compliance. The system's ability to process multiple data modalities and generate detailed probability distributions makes it particularly effective for identifying subtle patterns that might indicate potential manipulation or fraudulent activity.

### Exemplary Computing Environment

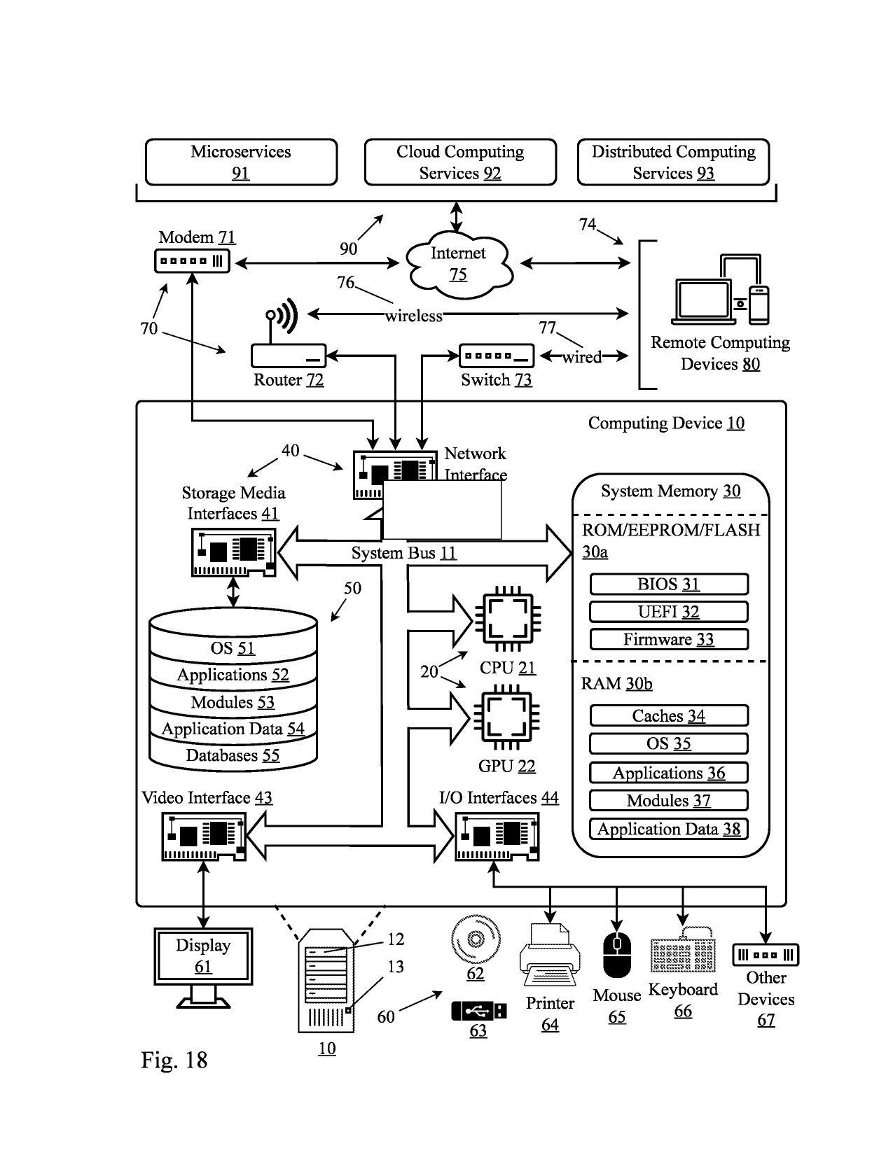

FIG. 18 illustrates an exemplary computing environment on which an embodiment described herein may be implemented, in full or in part. This exemplary computing environment describes computer-related components and processes supporting enabling disclosure of computer-implemented embodiments. Inclusion in this exemplary computing environment of well-known processes and computer components, if any, is not a suggestion or admission that any embodiment is no more than an aggregation of such processes or components. Rather, implementation of an embodiment using processes and components described in this exemplary computing environment will involve programming or configuration of such processes and components resulting in a machine specially programmed or configured for such implementation. The exemplary computing environment described herein is only one example of such an environment and other configurations of the components and processes are possible, including other relationships between and among components, and/or absence of some processes or components described. Further, the exemplary computing environment described herein is not intended to suggest any limitation as to the scope of use or functionality of any embodiment implemented, in whole or in part, on components or processes described herein.

The exemplary computing environment described herein comprises a computing device 10 (further comprising a system bus 11, one or more processors 20, a system memory 30, one or more interfaces 40, one or more non-volatile data storage devices 50), external peripherals and accessories 60, external communication devices 70, remote computing devices 80, and cloud- based services 90.

System bus 11 couples the various system components, coordinating operation of and data transmission between those various system components. System bus 11 represents one or more of any type or combination of types of wired or wireless bus structures including, but not limited to, memory busses or memory controllers, point-to-point connections, switching fabrics, peripheral busses, accelerated graphics ports, and local busses using any of a variety of bus architectures. By way of example, such architectures include, but are not limited to, Industry Standard Architecture (ISA) busses, Micro Channel Architecture (MCA) busses, Enhanced ISA (EISA) busses, Video Electronics Standards Association (VESA) local busses, a Peripheral Component Interconnects (PCI) busses also known as a Mezzanine busses, or any selection of, or combination of, such busses. Depending on the specific physical implementation, one or more of the processors 20, system memory 30 and other components of the computing device 10 can be physically co-located or integrated into a single physical component, such as on a single chip. In such a case, some or all of system bus 11 can be electrical pathways within a single chip structure.

Computing device may further comprise externally-accessible data input and storage devices 12 such as compact disc read-only memory (CD-ROM) drives, digital versatile discs (DVD), or other optical disc storage for reading and/or writing optical discs 62; magnetic cassettes, magnetic tape, magnetic disk storage, or other magnetic storage devices; or any other medium which can be used to store the desired content and which can be accessed by the computing device 10. Computing device may further comprise externally-accessible data ports or connections 12 such as serial ports, parallel ports, universal serial bus (USB) ports, and infrared ports and/or transmitter/receivers. Computing device may further comprise hardware for wireless communication with external devices such as IEEE 1394 ("Firewire") interfaces, IEEE 802.11 wireless interfaces, BLUETOOTH® wireless interfaces, and so forth. Such ports and interfaces may be used to connect any number of external peripherals and accessories 60 such as visual displays, monitors, and touch-sensitive screens 61, USB solid state memory data storage drives (commonly known as "flash drives" or "thumb drives") 63, printers 64, pointers and manipulators such as mice 65, keyboards 66, and other devices 67 such as joysticks and gaming pads, touchpads, additional displays and monitors, and external hard drives (whether solid state or disc-based), microphones, speakers, cameras, and optical scanners.

Processors 20 are logic circuitry capable of receiving programming instructions and processing (or executing) those instructions to perform computer operations such as retrieving data, storing data, and performing mathematical calculations. Processors 20 are not limited by the materials from which they are formed or the processing mechanisms employed therein, but are typically comprised of semiconductor materials into which many transistors are formed together into logic gates on a chip (i.e., an integrated circuit or IC). The term processor includes any device capable of receiving and processing instructions including, but not limited to, processors operating on the basis of quantum computing, optical computing, mechanical computing (e.g., using nanotechnology entities to transfer data), and so forth. Depending on configuration, computing device 10 may comprise more than one processor. For example, computing device 10 may comprise one or more central processing units (CPUs) 21, each of which itself has multiple processors or multiple processing cores, each capable of independently or semi-independently processing programming instructions based on technologies like complex instruction set computer (CISC) or reduced instruction set computer (RISC). Further, computing device 10 may comprise one or more specialized processors such as a graphics processing unit (GPU) 22 configured to accelerate processing of computer graphics and images via a large array of specialized processing cores arranged in parallel. Further computing device 10 may be comprised of one or more specialized processes such as Intelligent Processing Units, field- programmable gate arrays or application-specific integrated circuits for specific tasks or types of tasks. The term processor may further include: neural processing units (NPUs) or neural computing units optimized for machine learning and artificial intelligence workloads using specialized architectures and data paths; tensor processing units (TPUs) designed to efficiently perform matrix multiplication and convolution operations used heavily in neural networks and deep learning applications; application-specific integrated circuits (ASICs) implementing custom logic for domain-specific tasks; application-specific instruction set processors (ASIPs) with instruction sets tailored for particular applications; field-programmable gate arrays (FPGAs) providing reconfigurable logic fabric that can be customized for specific processing tasks; processors operating on emerging computing paradigms such as quantum computing, optical computing, mechanical computing (e.g., using nanotechnology entities to transfer data), and so forth. Depending on configuration, computing device 10 may comprise one or more of any of the above types of processors in order to efficiently handle a variety of general purpose and specialized computing tasks. The specific processor configuration may be selected based on performance, power, cost, or other design constraints relevant to the intended application of computing device 10.

System memory 30 is processor-accessible data storage in the form of volatile and/or nonvolatile memory. System memory 30 may be either or both of two types: non-volatile memory and volatile memory. Non-volatile memory 30a is not erased when power to the memory is removed, and includes memory types such as read only memory (ROM), electronically-erasable programmable memory (EEPROM), and rewritable solid state memory (commonly known as "flash memory"). Non-volatile memory 30a is typically used for long-term storage of a basic input/output system (BIOS) 31, containing the basic instructions, typically loaded during computer startup, for transfer of information between components within computing device, or a unified extensible firmware interface (UEFI), which is a modern replacement for BIOS that supports larger hard drives, faster boot times, more security features, and provides native support for graphics and mouse cursors. Non-volatile memory 30a may also be used to store firmware comprising a complete operating system 35 and applications 36 for operating computer-controlled devices. The firmware approach is often used for purpose-specific computer-controlled devices such as appliances and Internet-of-Things (IoT) devices where processing power and data storage space is limited. Volatile memory 30b is erased when power to the memory is removed and is typically used for short-term storage of data for processing. Volatile memory 30b includes memory types such as random-access memory (RAM), and is normally the primary operating memory into which the operating system 35, applications 36, program modules 37, and application data 38 are loaded for execution by processors 20. Volatile memory 30b is generally faster than non-volatile memory 30a due to its electrical characteristics and is directly accessible to processors 20 for processing of instructions and data storage and retrieval. Volatile memory 30b may comprise one or more smaller cache memories which operate at a higher clock speed and are typically placed on the same IC as the processors to improve performance.

There are several types of computer memory, each with its own characteristics and use cases. System memory 30 may be configured in one or more of the several types described herein, including high bandwidth memory (HBM) and advanced packaging technologies like chip-on-wafer-on-substrate (CoWoS). Static random access memory (SRAM) provides fast, low- latency memory used for cache memory in processors, but is more expensive and consumes more power compared to dynamic random access memory (DRAM). SRAM retains data as long as power is supplied. DRAM is the main memory in most computer systems and is slower than SRAM but cheaper and more dense. DRAM requires periodic refresh to retain data. NAND flash is a type of non-volatile memory used for storage in solid state drives (SSDs) and mobile devices and provides high density and lower cost per bit compared to DRAM with the trade-off of slower write speeds and limited write endurance. HBM is an emerging memory technology that provides high bandwidth and low power consumption which stacks multiple DRAM dies vertically, connected by through-silicon vias (TSVs). HBM offers much higher bandwidth (up to 1 TB/s) compared to traditional DRAM and may be used in high-performance graphics cards, Al accelerators, and edge computing devices. Advanced packaging and CoWoS are technologies that enable the integration of multiple chips or dies into a single package. CoWoS is a 2.5D packaging technology that interconnects multiple dies side-by-side on a silicon interposer and allows for higher bandwidth, lower latency, and reduced power consumption compared to traditional PCB-based packaging. This technology enables the integration of heterogeneous dies (e.g., CPU, GPU, HBM) in a single package and may be used in high-performance computing, Al accelerators, and edge computing devices.

Interfaces 40 may include, but are not limited to, storage media interfaces 41, network interfaces 42, display interfaces 43, and input/output interfaces 44. Storage media interface 41 provides the necessary hardware interface for loading data from non-volatile data storage devices 50 into system memory 30 and storage data from system memory 30 to non- volatile data storage device 50. Network interface 42 provides the necessary hardware interface for computing device 10to communicate with remote computing devices 80 and cloud-based services 90 via one or more external communication devices 70. Display interface 43 allows for connection of displays 61, monitors, touchscreens, and other visual input/output devices. Display interface 43 may include a graphics card for processing graphics-intensive calculations and for handling demanding display requirements. Typically, a graphics card includes a graphics processing unit (GPU) and video RAM (VRAM) to accelerate display of graphics. In some high- performance computing systems, multiple GPUs may be connected using NVLink bridges, which provide high-bandwidth, low-latency interconnects between GPUs. NVLink bridges enable faster data transfer between GPUs, allowing for more efficient parallel processing and improved performance in applications such as machine learning, scientific simulations, and graphics rendering. One or more input/output (I/O) interfaces 44 provide the necessary support for communications between computing device 10 and any external peripherals and accessories 60. For wireless communications, the necessary radio-frequency hardware and firmware may be connected to I/O interface 44 or may be integrated into I/O interface 44. Network interface 42 may support various communication standards and protocols, such as Ethernet and Small Form-Factor Pluggable (SFP). Ethernet is a widely used wired networking technology that enables local area network (LAN) communication. Ethernet interfaces typically use RJ45 connectors and support data rates ranging from 10 Mbps to 100 Gbps, with common speeds being 100 Mbps, 1 Gbps, 10 Gbps, 25 Gbps, 40 Gbps, and 100 Gbps. Ethernet is known for its reliability, low latency, and cost-effectiveness, making it a popular choice for home, office, and data center networks. SFP is a compact, hot-pluggable transceiver used for both telecommunication and data communications applications. SFP interfaces provide a modular and flexible solution for connecting network devices, such as switches and routers, to fiber optic or copper networking cables. SFP transceivers support various data rates, ranging from 100 Mbps to 100 Gbps, and can be easily replaced or upgraded without the need to replace the entire network interface card. This modularity allows for network scalability and adaptability to different network requirements and fiber types, such as single-mode or multi-mode fiber.

Non-volatile data storage devices 50 are typically used for long-term storage of data. Data on non-volatile data storage devices 50 is not erased when power to the non-volatile data storage devices 50 is removed. Non-volatile data storage devices 50 may be implemented using any technology for non-volatile storage of content including, but not limited to, CD-ROM drives, digital versatile discs (DVD), or other optical disc storage; magnetic cassettes, magnetic tape, magnetic disc storage, or other magnetic storage devices; solid state memory technologies such as EEPROM or flash memory; or other memory technology or any other medium which can be used to store data without requiring power to retain the data after it is written. Non-volatile data storage devices 50 may be non-removable from computing device 10 as in the case of internal hard drives, removable from computing device 10 as in the case of external USB hard drives, or a combination thereof, but computing device will typically comprise one or more internal, non- removable hard drives using either magnetic disc or solid state memory technology. Non-volatile data storage devices 50 may be implemented using various technologies, including hard disk drives (HDDs) and solid-state drives (SSDs). HDDs use spinning magnetic platters and read/write heads to store and retrieve data, while SSDs use NAND flash memory. SSDs offer faster read/write speeds, lower latency, and better durability due to the lack of moving parts, while HDDs typically provide higher storage capacities and lower cost per gigabyte. NAND flash memory comes in different types, such as Single-Level Cell (SLC), Multi-Level Cell (MLC), Triple-Level Cell (TLC), and Quad-Level Cell (QLC), each with trade-offs between performance, endurance, and cost. Storage devices connect to the computing device 10 through various interfaces, such as SATA, NVMe, and PCIe. SATA is the traditional interface for HDDs and SATA SSDs, while NVMe (Non-Volatile Memory Express) is a newer, high-performance protocol designed for SSDs connected via PCIe. PCIe SSDs offer the highest performance due to the direct connection to the PCIe bus, bypassing the limitations of the SATA interface. Other storage form factors include M.2 SSDs, which are compact storage devices that connect directly to the motherboard using the M.2 slot, supporting both SATA and NVMe interfaces. Additionally, technologies like Intel Optane memory combine 3D XPoint technology with NAND flash to provide high-performance storage and caching solutions. Non-volatile data storage devices 50 may be non-removable from computing device 10, as in the case of internal hard drives, removable from computing device 10, as in the case of external USB hard drives, or a combination thereof. However, computing devices will typically comprise one or more internal, non-removable hard drives using either magnetic disc or solid-state memory technology. Non-volatile data storage devices 50 may store any type of data including, but not limited to, an operating system 51 for providing low-level and mid-level functionality of computing device 10, applications 52 for providing high-level functionality of computing device 10, program modules 53 such as containerized programs or applications, or other modular content or modular programming, application data 54, and databases 55 such as relational databases, non-relational databases, object oriented databases, NoSQL databases, vector databases, knowledge graph databases, key-value databases, document oriented data stores, and graph databases.

Applications (also known as computer software or software applications) are sets of programming instructions designed to perform specific tasks or provide specific functionality on a computer or other computing devices. Applications are typically written in high-level programming languages such as C, C++, Scala, Erlang, GoLang, Java, Scala, Rust, and Python, which are then either interpreted at runtime or compiled into low-level, binary, processor- executable instructions operable on processors 20. Applications may be containerized so that they can be run on any computer hardware running any known operating system. Containerization of computer software is a method of packaging and deploying applications along with their operating system dependencies into self-contained, isolated units known as containers. Containers provide a lightweight and consistent runtime environment that allows applications to run reliably across different computing environments, such as development, testing, and production systems facilitated by specifications such as containerd.

The memories and non-volatile data storage devices described herein do not include communication media. Communication media are means of transmission of information such as modulated electromagnetic waves or modulated data signals configured to transmit, not store, information. By way of example, and not limitation, communication media includes wired communications such as sound signals transmitted to a speaker via a speaker wire, and wireless communications such as acoustic waves, radio frequency (RF) transmissions, infrared emissions, and other wireless media.

External communication devices 70 are devices that facilitate communications between computing device and either remote computing devices 80, or cloud-based services 90, or both. External communication devices 70 include, but are not limited to, data modems 71 which facilitate data transmission between computing device and the Internet 75 via a common carrier such as a telephone company or internet service provider (ISP), routers 72 which facilitate data transmission between computing device and other devices, and switches 73 which provide direct data communications between devices on a network or optical transmitters (e.g., lasers). Here, modem 71 is shown connecting computing device 10 to both remote computing devices 80 and cloud-based services 90 via the Internet 75. While modem 71, router 72, and switch 73 are shown here as being connected to network interface 42, many different network configurations using external communication devices 70 are possible. Using external communication devices 70, networks may be configured as local area networks (LANs) for a single location, building, or campus, wide area networks (WANs) comprising data networks that extend over a larger geographical area, and virtual private networks (VPNs) which can be of any size but connect computers via encrypted communications over public networks such as the Internet 75. As just one exemplary network configuration, network interface 42 may be connected to switch 73 which is connected to router 72 which is connected to modem 71 which provides access for computing device 10 to the Internet 75. Further, any combination of wired 77 or wireless 76 communications between and among computing device 10, external communication devices 70, remote computing devices 80, and cloud-based services 90 may be used. Remote computing devices 80, for example, may communicate with computing device through a variety of communication channels 74 such as through switch 73 via a wired 77 connection, through router 72 via a wireless connection 76, or through modem 71 via the Internet 75. Furthermore, while not shown here, other hardware that is specifically designed for servers or networking functions may be employed. For example, secure socket layer (SSL) acceleration cards can be used to offload SSL encryption computations, and transmission control protocol/internet protocol (TCP/IP) offload hardware and/or packet classifiers on network interfaces 42 may be installed and used at server devices or intermediate networking equipment (e.g., for deep packet inspection).

In a networked environment, certain components of computing device 10 may be fully or partially implemented on remote computing devices 80 or cloud-based services 90. Data stored in non-volatile data storage device 50 may be received from, shared with, duplicated on, or offloaded to a non-volatile data storage device on one or more remote computing devices 80 or in a cloud computing service 92. Processing by processors 20 may be received from, shared with, duplicated on, or offloaded to processors of one or more remote computing devices 80 or in a distributed computing service 93. By way of example, data may reside on a cloud computing service 92, but may be usable or otherwise accessible for use by computing device 10. Also, certain processing subtasks may be sent to a microservice 91 for processing with the result being transmitted to computing device 10 for incorporation into a larger processing task. Also, while components and processes of the exemplary computing environment are illustrated herein as discrete units (e.g., OS 51 being stored on non-volatile data storage device 51 and loaded into system memory 35 for use) such processes and components may reside or be processed at various times in different components of computing device 10, remote computing devices 80, and/or cloud-based services 90. Also, certain processing subtasks may be sent to a microservice 91 for processing with the result being transmitted to computing device 10 for incorporation into a larger processing task. Infrastructure as Code (IaaC) tools like Terraform can be used to manage and provision computing resources across multiple cloud providers or hyperscalers. This allows for workload balancing based on factors such as cost, performance, and availability. For example, Terraform can be used to automatically provision and scale resources on AWS spot instances during periods of high demand, such as for surge rendering tasks, to take advantage of lower costs while maintaining the required performance levels. In the context of rendering, tools like Blender can be used for object rendering of specific elements, such as a car, bike, or house. These elements can be approximated and roughed in using techniques like bounding box approximation or low-poly modeling to reduce the computational resources required for initial rendering passes. The rendered elements can then be integrated into the larger scene or environment as needed, with the option to replace the approximated elements with higher-fidelity models as the rendering process progresses.

In an implementation, the disclosed systems and methods may utilize, at least in part, containerization techniques to execute one or more processes and/or steps disclosed herein. Containerization is a lightweight and efficient virtualization technique that allows you to package and run applications and their dependencies in isolated environments called containers. One of the most popular containerization platforms is containerd, which is widely used in software development and deployment. Containerization, particularly with open-source technologies like containerd and container orchestration systems like Kubernetes, is a common approach for deploying and managing applications. Containers are created from images, which are lightweight, standalone, and executable packages that include application code, libraries, dependencies, and runtime. Images are often built from a containerfile or similar, which contains instructions for assembling the image. Containerfiles are configuration files that specify how to build a container image. Systems like Kubernetes natively support containerd as a container runtime. They include commands for installing dependencies, copying files, setting environment variables, and defining runtime configurations. Container images can be stored in repositories, which can be public or private. Organizations often set up private registries for security and version control using tools such as Harbor, JFrog Artifactory and Bintray, GitLab Container Registry, or other container registries. Containers can communicate with each other and the external world through networking. Containerd provides a default network namespace, but can be used with custom network plugins. Containers within the same network can communicate using container names or IP addresses.

Remote computing devices 80 are any computing devices not part of computing device 10. Remote computing devices 80 include, but are not limited to, personal computers, server computers, thin clients, thick clients, personal digital assistants (PDAs), mobile telephones, watches, tablet computers, laptop computers, multiprocessor systems, microprocessor based systems, set-top boxes, programmable consumer electronics, video game machines, game consoles, portable or handheld gaming units, network terminals, desktop personal computers (PCs), minicomputers, mainframe computers, network nodes, virtual reality or augmented reality devices and wearables, and distributed or multi-processing computing environments. While remote computing devices 80 are shown for clarity as being separate from cloud-based services 90, cloud-based services 90 are implemented on collections of networked remote computing devices 80.

Cloud-based services 90 are Internet-accessible services implemented on collections of networked remote computing devices 80. Cloud-based services are typically accessed via application programming interfaces (APIs) which are software interfaces which provide access to computing services within the cloud-based service via API calls, which are pre-defined protocols for requesting a computing service and receiving the results of that computing service. While cloud-based services may comprise any type of computer processing or storage, three common categories of cloud-based services 90 are serverless logic apps, microservices 91, cloud computing services 92, and distributed computing services 93.

Microservices 91 are collections of small, loosely coupled, and independently deployable computing services. Each microservice represents a specific computing functionality and runs as a separate process or container. Microservices promote the decomposition of complex applications into smaller, manageable services that can be developed, deployed, and scaled independently. These services communicate with each other through well-defined application programming interfaces (APIs), typically using lightweight protocols like HTTP, protobuffers, gRPC or message queues such as Kafka. Microservices 91 can be combined to perform more complex or distributed processing tasks. In an embodiment, Kubernetes clusters with containerized resources are used for operational packaging of system.

Cloud computing services 92 are delivery of computing resources and services over the Internet 75 from a remote location. Cloud computing services 92 provide additional computer hardware and storage on as-needed or subscription basis. Cloud computing services 92 can provide large amounts of scalable data storage, access to sophisticated software and powerful server-based processing, or entire computing infrastructures and platforms. For example, cloud computing services can provide virtualized computing resources such as virtual machines, storage, and networks, platforms for developing, running, and managing applications without the complexity of infrastructure management, and complete software applications over public or private networks or the Internet on a subscription or alternative licensing basis, or consumption or ad-hoc marketplace basis, or combination thereof.

Federated distributed computing services 93 provide large-scale processing using multiple interconnected computers or nodes to solve computational problems or perform tasks collectively. In federated distributed computing, the processing and storage capabilities of multiple machines are leveraged to work together as a unified system, even when different tiers or tesselations may have limited or even no visibility into the resources and processing layer up or downstream. Federated distributed computing services are designed to address problems that cannot be efficiently solved by a single computer or that require large-scale computational power and require dynamism and workload distribution for economic, security or privacy reasons not well supported by canonical distributed computing resources; e.g. most commonly cloud-based computing applications, resources or analytics. Federated DCG coordinated variants of these services enable superior decentralization and further enhance parallel processing, fault tolerance, and scalability by distributing tasks across multiple tiers or tesselations while enabling computing process dependency calculation with varying degrees of visibility, assurance and privacy or security based on constituent computing system, network, workload and user or provider needs and preferences as well as practical legal and regulatory concerns to include but not limited to data localization, national data transfer restrictions, privacy and consumer protections, wiretap/telecommunications monitoring requirements, encryption and data routing and intermediate processing restrictions.

Although described above as a physical device, computing device 10 can be a virtual computing device, in which case the functionality of the physical components herein described, such as processors 20, system memory 30, network interfaces 40, and other like components can be provided by computer-executable instructions. Such computer-executable instructions can execute on a single physical computing device, or can be distributed across multiple physical computing devices, including being distributed across multiple physical computing devices in a dynamic manner such that the specific, physical computing devices hosting such computer- executable instructions can dynamically change over time depending upon need and availability. In the situation where computing device 10 is a virtualized device, the underlying physical computing devices hosting such a virtualized computing device can, themselves, comprise physical components analogous to those described above, and operating in a like manner. Furthermore, virtual computing devices can be utilized in multiple layers with one virtual computing device executing within the construct of another virtual computing device. Thus, computing device 10 may be either a physical computing device or a virtualized computing device within which computer-executable instructions can be executed in a manner consistent with their execution by a physical computing device. Similarly, terms referring to physical components of the computing device, as utilized herein, mean either those physical components or virtualizations thereof performing the same or equivalent functions.

The skilled person will be aware of a range of possible modifications of the various aspects described above. Accordingly, the present invention is defined by the claims and their equivalents.

What is claimed is:

1. A computing system for a sports betting and gaming platform with integrated AI-enabled fraud detection, the computing system comprising:
one or more hardware processors configured for:
training a network of machine learning models, wherein each machine learning model is specialized for a particular sport;
processing a plurality of sport and player data through a trained machine learning model;
generating a player profile and a betting profile based on the sport and player data;
measure how far the player profile or the betting profile deviates from an expected profile which includes a deviation threshold;
generating a fraud alert when the measured deviation passes the deviation threshold; and
transmitting the fraud alert to a regulator.

2. The computing system of claim 1, wherein the sport and player data comprises game telemetry, betting history, social media content, biometric information, and facility-specific environmental factors.

3. The computing system of claim 1, wherein the profiles comprise performance metrics, team dynamics, historical patterns, and network interaction analysis across multiple competition levels.

4. The computing system of claim 1, wherein the one or more hardware processors are further configured for:
accessing a personal health database (PHDB) containing historical health records, injury data, and recovery patterns;
preprocessing the PHDB information to standardize health metrics across different sports and competition levels;
processing the preprocessed PHDB information through the trained machine learning models and incorporating the data into at least one of the player profile or betting profile.

5. The computing system of claim 1, wherein the network of machine learning models includes generative adversarial networks, temporal convolutional networks, and graph attention networks.

6. A computer-implemented method for a sports betting and gaming platform with integrated AI-enabled fraud detection, the computer-implemented method comprising:
training a network of machine learning models, wherein each machine learning model is specialized for a particular sport;
processing a plurality of sport and player data through a trained machine learning model;
generating a player profile and a betting profile based on the sport and player data;
measuring how far the player profile or the betting profile deviates from an expected profile which includes a deviation threshold;
generating a fraud alert when the measured deviation passes the deviation threshold; and
transmitting the fraud alert to a regulator.

7. The computer-implemented method of claim 6, wherein the sport and player data comprises game telemetry, betting history, social media content, biometric information, and facility-specific environmental factors.

8. The computer-implemented method of claim 6, wherein the profiles comprise performance metrics, team dynamics, historical patterns, and network interaction analysis across multiple competition levels.

9. The computer-implemented method of claim 6, further comprising:
accessing a personal health database (PHDB) containing historical health records, injury data, and recovery patterns;
preprocessing the PHDB information to standardize health metrics across different sports and competition levels;
processing the preprocessed PHDB information through the trained machine learning models and incorporating the data into at least one of the player profile or betting profile.

10. The computer-implemented method of claim 6, wherein the network of machine learning models includes generative adversarial networks, temporal convolutional networks, and graph attention networks.

11. A system for a sports betting and gaming platform with integrated AI-enabled fraud detection, comprising one or more computers with executable instructions that, when executed, cause the system to:
train a network of machine learning models, wherein each machine learning model is specialized for a particular sport;
process a plurality of sport and player data through a trained machine learning model;
generate a player profile and a betting profile based on the sport and player data;
measure how far the player profile or the betting profile deviates from an expected profile which includes a deviation threshold;
generate a fraud alert when the measured deviation passes the deviation threshold; and
transmit the fraud alert to a regulator.

12. The system of claim 11, wherein the sport and player data comprises game telemetry, betting history, social media content, biometric information, and facility-specific environmental factors.

13. The system of claim 11, wherein the profiles comprise performance metrics, team dynamics, historical patterns, and network interaction analysis across multiple competition levels.

14. The system of claim 11, wherein the one or more computers with executable instructions that, when executed, further cause the system to:
access a personal health database (PHDB) containing historical health records, injury data, and recovery patterns;
preprocess the PHDB information to standardize health metrics across different sports and competition levels;
process the preprocessed PHDB information through the trained machine learning models and incorporating the data into at least one of the player profile or betting profile.

15. The system of claim 11, wherein the network of machine learning models includes generative adversarial networks, temporal convolutional networks, and graph attention networks.
